(19) United States
(12) Patent Application Publication
(10) Pub. No.: US 2003/0077645 A1
(43) Pub. Date: Apr. 24, 2003

(54) Superfamily receptor chimeras, translocation assay for superfamily receptor ligands, and methods and kits for detecting and characterizing receptor ligands

(76) Inventors: Gordon L. Hager, Garrett Park, MD (US); Susan MacKem, Bethesda, MD (US)

Correspondence Address: NATH & ASSOCIATES, 1030 15th STREET, 6TH FLOOR, WASHINGTON, DC, 20005, US

(21) Appl. No.: 10/259,864

(22) Filed: Sep. 30, 2002

Related U.S. Application Data
(60) Provisional application No. 60/325,178, filed on Sep. 28, 2001.

(51) Int. Cl. C12Q 1/68; C07H 21/04; C12P 21/02; C12N 5/06; C07K 14/72

(52) U.S. Cl. 435/6; 435/69.1; 435/320.1; 435/325; 530/350; 536/23.5

(57) ABSTRACT

The present invention relates to novel superfamily receptor chimeras, methods for using superfamily receptor chimeras, methods and kits for detecting cellular function and metabolic state, and the treatment of disease states involving defective receptor protein cytoplasm/nuclear translocation. In particular, this invention is preferably directed to chimeras of glucocorticoid receptor/superfamily receptor proteins.

This application claims the benefit of U.S. Provisional Patent Application No. 60/325,178, filed Sep. 28, 2001, the contents of which is hereby incorporated by reference in its entirety.

### BACKGROUND OF THE INVENTION

1. Field of Invention

The present invention relates to novel superfamily receptor chimeras, methods for using superfamily receptor chimeras, methods and kits for detecting cellular function and metabolic state, and the treatment of disease states involving defective receptor protein cytoplasm/nuclear translocation. In particular, this invention is preferably directed to chimeras of glucocorticoid receptor/superfamily receptor proteins.

2. Background

Steroid receptors mediate the action of a broad range of low molecular weight ligands, commonly referred to as hormones. These molecules influence a wide variety of human physiological processes, and also impact many human disease states, including cancer, heart disease, arteriosclerosis, arthritis, and inflammatory states. Low molecular weight hormones diffuse throughout the body and pass through cell membranes, either in a passive mode, or in some cases via active transport. Once in a cell, each hormone will interact with a cognate receptor; the receptor for each ligand recognizes and binds with a high affinity to its cognate hormone. This specific binding event induces conformational changes in the structure of the receptor, and activates the receptor for a variety of intracellular processes. The primary mechanism by which receptors act is by binding to specific regulatory sites in chromosomes, and regulating the rate of expression of target cellular genes. A secondary mechanism, referred to as “rapid” or “non-genomic” events, involves the interaction with specific cellular protein kinase signaling cascades.

The steroid, nuclear, and orphan receptor family now encompasses a superfamily of proteins, with approximately 70-75 distinct members in the mammalian species. Some members of this family do not have recognized cognate ligands, and thus are referred to as “orphan” receptors. A second group, characterized originally as “nuclear” receptors, are located almost exclusively in the nucleus, both in the presence and the absence of ligand. A third group display both cytoplasmic and nuclear distributions, although the precise partitioning may change with the addition of hormone.

The subcellular distribution of these receptors clearly plays a major role in their biological activity. Many receptors, such as the glucocorticoid receptor (“GR”), are in the cytoplasm in the absence of ligand, and thus do not interact with target genes. For those receptors that undergo a large-scale cytoplasmic to nuclear translocation, an assay of the compartmental distribution provides a potentially useful method to detect the presence of the cognate ligand in a given biological sample. There is a need for improved assays for ligand detection.

Many chimeric receptors have been described, but these fusions have been characterized and considered exclusively in terms of their gene activation potential (see, for example, Retinoid-X Receptor Signalling in the Developing Spinal Cord, Solomin, L., Johansson, C. B., Zetterstrom, R. H., Bissonnette, R. P., Heyman, R. A., Olson, L., Lendahl, U., Frisen, J., and Perlmann, T., Nature 395:398-402 (1998).).

Further, few steroid, nuclear, or orphan receptors manifest a distinct cytoplasmic to nuclear translocation in response to hormone stimulation. The vast majority of the molecules distribute to the nucleus, or in a mixed cytoplasmic/nuclear arrangement. Thus, use of a subcellular distribution assay to monitor hormone function has not heretofore been possible.

Applicants have solved these problems by the construction of a chimeric receptor, comprising the N-terminal domain of the glucocorticoid receptor and the C-terminal, or ligand binding, domain of a target superfamily receptor. The inventive receptor retains the cytoplasmic/nuclear translocation properties of the glucocorticoid receptor, but responds only to a ligand for the target receptor.

We describe here chimeric proteins that reside in the cytoplasm of untreated cells, but manifest complete translocation to the nucleus when cells are induced with a ligand for the ligand-binding domain of the chimeric receptor protein. The inventive chimeras form the basis for a new, in vivo translocation assay for superfamily receptor ligands, and has important implications for mechanisms of receptor subcellular trafficking.

Further, the inventive subject matter opens a completely new approach to the study of the function of superfamily receptors. In particular, when labeled with an appropriate fluorescent tag, such chimeric receptors permit the use of a real time subcellular translocation assay to monitor the presence and concentration of the cognate ligand in living cells in real time. The inventive cell-based assay provides a direct method to detect the presence and concentration of a given hormone or other ligand in a sample. Further, the inventive methods provide a less expensive and complicated approach to the process of screening for ligands for the orphan receptors and alternate ligands for the characterized receptors. The availability of chimeric proteins with the nuclear/cytoplasmic translocation properties of GR, and the ligand responsiveness of a superfamily receptor, provides a powerful translocation assay for known superfamily receptor ligands, for the identification of novel ligand analogues, and for the identification of ligands for orphan receptors.

### SUMMARY OF THE INVENTION

The present invention relates to a method for making a recombinant nuclear translocation protein, comprising:

covalently connecting (i) a glucocorticoid receptor DNA sequence coding for the cytoplasmic/nuclear translocation domain of the glucocorticoid receptor protein, (ii) a superfamily receptor DNA sequence coding for the ligand binding domain of a superfamily receptor protein, and (iii) a nucleic acid sequence for a marker protein domain, to form a DNA chimera,

wherein said superfamily receptor DNA sequence is connected to the 3′ end of said glucocorticoid receptor DNA sequence; and

expressing said DNA chimera in an expression system to prepare said protein.

The present invention further relates to a protein produced by the process of:

covalently connecting (i) a glucocorticoid receptor DNA sequence coding for the 540 end of the DNA sequence of the glucocorticoid receptor protein, through and including the complete nuclear localization sequence and the complete helix 1 sequence of said glucocorticoid receptor DNA sequence, (ii) a superfamily receptor DNA sequence coding for the 3′ end of the DNA sequence of a superfamily receptor protein, through and including the complete ligand binding domain sequence and the complete helix 3 sequence of said superfamily receptor DNA sequence, and (iii) a nucleic acid sequence for a marker protein domain, to form a DNA chimera,

wherein said superfamily receptor DNA sequence is connected to the 3′ end of said glucocorticoid receptor DNA sequence,

wherein said marker protein domain DNA sequence is covalently connected to the 5′ end of said glucocorticoid receptor DNA sequence,

and wherein said translocation domain of the glucocorticoid receptor and said ligand binding domain of a superfamily receptor are covalently connected by a DNA linker sequence; and

expressing said DNA chimera in an expression system to prepare said protein.

The present invention further relates to a nucleic acid chimera comprising:

a nucleic acid sequence which codes for the cytoplasmic/nuclear translocation domain of glucocorticoid receptor protein; and

a nucleic acid sequence which codes for the ligand binding domain of a superfamily receptor protein.

The present invention further relates to a chimeric protein comprising two elements:

a glucocorticoid receptor 5′ end, encompassing the nuclear translocation domain and helix 1; and

a superfamily receptor 3′ end, encompassing the ligand binding domain and helix 3.

The present invention further relates to a method for detecting a ligand of a superfamily receptor protein, which comprises:

producing a nucleic acid vector encoding a nucleic acid chimera comprising three elements: a 5′ end of a glucocorticoid receptor, encompassing the nuclear translocation domain and helix 1, a 3′ end of a superfamily receptor, encompassing the ligand binding domain and helix 3, and a nucleic acid sequence for a marker protein domain;

transfecting a eukaryotic cell with said nucleic acid vector;

isolating a clonal population of cells that express a chimeric protein translated from said nucleic acid vector;

contacting said cells with a sample compound or composition; and

detecting the presence of cytoplasmic/nuclear translocation in response to a ligand of said ligand binding domain.

The present invention further relates to a method for determining the concentration of a ligand of a labeled chimeric superfamily receptor protein, which comprises:

producing a nucleic acid vector encoding a nucleic acid chimera comprising: a nucleic acid sequence coding for the cytoplasmic/nuclear translocation domain of glucocorticoid receptor protein, a nucleic acid sequence coding for the ligand binding domain of a superfamily receptor protein, and a nucleic acid sequence for a marker protein domain;

transfecting a eukaryotic cell with said nucleic acid vector;

isolating a clonal population of transfected cells that express a chimeric protein translated from said nucleic acid vector;

contacting said transfected cells with a sample;

scanning one or more test cell(s) to obtain signal data from said labeled protein;

converting said signal data to obtain the cellular location of said labeled protein in said test cell(s); and

analyzing said data using an analysis system having an algorithm to calculate changes in the distribution of said labeled protein between the cell cytoplasm and the cell nucleus of said test cell(s), said analysis system having the capability of providing an accurate reading of the concentration of a ligand.

Further, the present invention relates to a kit for detecting and screening for a ligand of a superfamily receptor protein in an environmental sample, comprising:

a live-cell system which expresses a chimeric protein comprising the cytoplasmic/nuclear translocation domain of glucocorticoid receptor protein, the ligand binding domain of a superfamily receptor protein, and a marker protein domain; and

a detection system for the detection of the translocation of said marker protein.

The present invention further relates to a kit for detecting and screening for a ligand of a superfamily receptor protein in an environmental sample, comprising:

a quantity of a chimeric protein comprising the cytoplasmic/nuclear translocation domain of glucocorticoid receptor protein, the ligand binding domain of a superfamily receptor protein, and a marker protein domain;

a cell-free membrane system which restricts translocation of the chimeric protein when no ligand is bound to the ligand binding domain of said chimeric protein, and which permits translocation of the chimeric protein when the ligand binding domain of said chimeric protein is bound to its ligand; and

a detection system for the detection of the translocation of said marker protein.

The present invention further relates to a method for diagnosis of defects in the nuclear transportation process, which comprises:

producing a nucleic acid vector encoding a nucleic acid chimera comprising: a nucleic acid sequence coding for the cytoplasmic/nuclear translocation domain of glucocorticoid receptor protein, a nucleic acid sequence coding for the ligand binding domain of a superfamily receptor protein, and a nucleic acid sequence for a marker protein domain;

transfecting a set of suspected defective cells with said nucleic acid vector;

isolating a clonal population of said cells that express a chimeric protein translated from said nucleic acid vector;

contacting said cells with a ligand of said ligand binding domain; and

detecting the presence or absence of cytoplasmic/nuclear translocation in response to said ligand.

Finally, the present invention further relates to a method for treating defective translocation of a superfamily receptor protein from the cytoplasm to the nucleus of a cell, in an animal in need thereof, comprising:

producing a nucleic acid vector which is capable of being transcribed, and which encodes a nucleic acid chimera comprising: a nucleic acid sequence coding for the cytoplasmic/nuclear translocation domain of glucocorticoid receptor protein and a nucleic acid sequence coding for the ligand binding domain of said superfamily receptor protein;

transfecting a target cell in said animal with said nucleic acid vector.

### BRIEF DESCRIPTION OF THE DRAWINGS

FIG. 1 is a drawing which depicts the construction of a chimeric GFP-GR-RAR receptor.

FIG. 2 is a graph which depicts transcriptional transactivation by a chimeric GFP-GR-RAR receptor.

FIG. 3 is a series of photographs which depict subcellular trafficking of a chimeric GFP-GR-RAR receptor.

FIG. 4 is a photograph which depicts the interaction of a chimeric GFP-GR-RAR receptor with heat shock protein 90.

FIG. 5 is a drawing which depicts the construction of a chimeric GFP-GR-ER receptor.

FIG. 6 is a series of photographs which depict subcellular trafficking of wild type ER receptor.

FIG. 7 is a series of photographs which depict subcellular trafficking of a chimeric GFP-GR-ER receptor.

FIG. 8 is a chart which depicts sequence alignments of the DNA sequence surrounding helix 1 and helix 3 in several representative mammalian steroid receptor DNA sequences.

FIG. 9 is a chart which depicts sequence alignments of the DNA sequence surrounding helix 3 in 49 mammalian steroid receptor DNA sequences.

FIG. 10 is a drawing which depicts the relationship between the protein secondary structure domains in four chimeric molecules synthesized by Applicants.

### DETAILED DESCRIPTION OF THE INVENTION

The inventive subject matter opens a completely new approach to the study of the function of superfamily receptors. In particular, when labeled with an appropriate fluorescent tag, such chimeric receptors permit the use of a real time subcellular translocation assay to monitor the presence and concentration of the cognate ligand in living cells in real time. The inventive cell-based assay provides a direct method to detect the presence and concentration of a given hormone or other ligand in a sample. Further, the inventive methods provide a less expensive and complicated approach to the process of screening for ligands for the orphan receptors and alternate ligands for the characterized receptors. The availability of chimeric proteins with the nuclear/cytoplasmic translocation properties of GR, and the ligand responsiveness of a superfamily receptor, provides a powerful translocation assay for known superfamily receptor ligands, for the identification of novel ligand analogues, and for the identification of ligands for orphan receptors.

### Definitions

“Chimera” refers to a recombinant nucleic acid molecule generated by cloning portion(s) of one or more nucleic acid sequence(s) in-frame into one or more other nucleic acid sequence(s) to produce a single nucleic acid sequence capable of being transcribed into a polypeptide. The polypeptide produced by such a nucleic acid sequence chimera is referred to as a “chimeric protein” or “protein chimera”.

“Superfamily receptor” refers to the complete family of steroid, nuclear, and orphan receptor proteins having an identifiable ligand binding domain. The term is intended to encompass the known classic nuclear receptors, hormone receptors, and orphan receptors, as well as proteins having an identifiable ligand binding domain which may discovered in the future.

“GR” refers to the glucocorticoid receptor protein.

“ER” the estrogen receptor protein α.

“GFP” refers to green fluorescent protein.

“Marker protein domain” refers to a protein domain which is detectable based on inherent structural or functional characteristics, such as fluorescence. In this regard, a number of fluorescent proteins of various colors such as yellow, green, red, and blue are known, and are contemplated by this definition.

“LBD” refers to a ligand binding domain of a receptor protein.

“GR*” refers to a mutant GR having increased ligand binding affinity and which is fused to GFP.

“RAR” refers to the retinoic acid receptor.

“GR-RAR” refers to a chimera which encodes GR* and its associated GFP, wherein the LBD of wild-type GR is replaced by the LBD of RAR.

“ATRA” refers to all-trans retinoic acid.

“Dex” refers to dexamethasone.

“Hsp” refers to heat shock protein.

“Environmental sample” refers to a compound or composition to be screened for the presence or concentration of a ligand, and includes, without limitation, blood, serum, and tissue samples, as well as individual samples or libraries thereof.

A “cell-based” system is a system which is based upon the use of cells derived, isolated, or otherwise acquired from a living organism, and includes, for example, a blood or serum sample and a cell culture.

“Wild-type nucleic acid sequence” refers to the nucleic acid sequence(s) found in subjects having normal function in the protein transcribed from the nucleic acid sequence(s). It is to be understood that some variation in wild-type nucleic acid sequence is to be expected, and the salient feature is normal protein function. “Wild-type” nucleic acid sequence is to be distinguished from nucleic acid sequence(s) transcribing defective or loss-of-function proteins.

A nucleic acid sequence “capable of being transcribed” refers to a nucleic acid sequence which, when introduced into a target cell, forms a complete transcription complex. The resulting complete transcription complex may comprise the required elements as introduced with the exogenous nucleic acid sequence, as provided by the target cell, or a combination of introduced and existing elements.

“Isolated nucleic acid sequence” refers to a nucleic acid sequence which has been purified to at least about 95% homogeneity. This definition includes nucleic acid sequences that hybridize under stringent hybridization conditions with a gene, such as a cDNA molecule. This definition further includes a genomic sequence of interest comprising a nucleic acid sequence between the initiation codon and the stop codon, including all of the introns that are normally present in a native chromosome. It may further include the 3′ and 5′ untranslated regions found in a mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kb or smaller; and substantially free of flanking chromosomal sequence. The genomic DNA flanking the coding region, either 3′ or 5′, or internal regulatory sequences as sometimes found in introns, contains sequences required for proper tissue and stage specific expression.

In the context of interactions with a chimeric protein of the present invention, “associate” refers to a stable covalent or non-covalent bond which exists between said chimeric protein in the absence of the ligand for the LBD of said receptor protein.

“Cell-free membrane system” refers to a membrane which is either a product of a living cell or tissue and has been isolated outside a living cell, or is not the product of a living cell or tissue.

“Effecting” refers to the process of producing an effect on biological activity, function, health, or condition of an organism in which such biological activity, function, health, or condition is maintained, enhanced, diminished, or treated in a manner which is consistent with the general health and well-being of the organism.

“Enhancing” the biological activity, function, health, or condition of an organism refers to the process of augmenting, fortifying, strengthening, or improving.

“Pharmaceutically acceptable salt, ester, or solvate” refers to a salt, ester, or solvate of a subject compound which possesses the desired pharmacological activity and which is neither biologically nor otherwise undesirable. A salt, ester, or solvate can be formed with inorganic acids such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, gluconate, glycerophosphate, hemisulfate, heptanoate, hexanoate, hydrochloride, hydrobromide, hydroiodide, 2-hydroxyethanesulfonate, lactate, maleate, methanesulfonate, naphthylate, 2-naphthalenesulfonate, nicotinate, oxalate, sulfate, thiocyanate, tosylate and undecanoate. Examples of base salts, esters, or solvates include ammonium salts; alkali metal salts, such as sodium and potassium salts; alkaline earth metal salts, such as calcium and magnesium salts; salts with organic bases, such as dicyclohexylamine salts; N-methyl-D-glucamine; and salts with amino acids, such as arginine, lysine, and so forth. Also, the basic nitrogen-containing groups can be quarternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; aralkyl halides, such as benzyl and phenethyl bromides; and others. Water or oil-soluble or dispersible products are thereby obtained.

### Methods for Making Chimeric Receptors

Multicellular organisms require specific intercellular communication to organize the complex body plan properly during embryogenesis, and to maintain its properties and functions during the entire life span. Small, hydrophobic signaling molecules, such as steroid hormones, certain vitamins, and metabolic intermediates, enter or are generated within target cells and bind to cognate members of a large family of nuclear receptors. Nuclear receptors (NRs) are of major importance for metazoan intercellular signaling, as they bring together different intracellular and extracellular signals to initiate and regulate gene expression programs. They act as transcription factors that: (1) respond directly through physical association with a large variety of hormonal and other regulatory, as well as metabolic signals; (2) integrate diverse signaling pathways as they are themselves targets of posttranslational modifications; and (3) regulate the activities of other signaling cascades, commonly referred to as signal transduction cross-talk. The genetic programs that they modulate affect virtually all aspects of the life of a multicellular organism, covering such diverse aspects as, for example, embryogenesis, homeostasis and reproduction, or cell growth and death.

Thus, there is a great need for both a better understanding of the functioning of superfamily receptors, and especially for the ability to successfully manipulate superfamily receptors to produce chimeric proteins useful for diagnostic and treatment purposes. With these needs in mind, the present invention relates to a method for making a recombinant nuclear translocation protein, comprising:

covalently connecting (i) a glucocorticoid receptor DNA sequence coding for the cytoplasmic/nuclear translocation domain of the glucocorticoid receptor protein, (ii) a superfamily receptor DNA sequence coding for the ligand binding domain of a superfamily receptor protein, and (iii) a nucleic acid sequence for a marker protein domain, to form a DNA chimera,

wherein said superfamily receptor DNA sequence is connected to the 3′ end of said glucocorticoid receptor DNA sequence; and

expressing said DNA chimera in an expression system to prepare said protein.

In a preferred embodiment, said method further comprises covalently connecting said translocation domain of the glucocorticoid receptor and said ligand binding domain of a superfamily receptor utilizing a DNA linker sequence.

In a more preferred embodiment, said DNA linker sequence is a fragment of a polylinker sequence.

In another preferred embodiment, said marker protein domain DNA sequence is covalently connected to the 5′ end of said glucocorticoid receptor DNA sequence.

In another preferred embodiment, said method further comprises covalently connecting said glucocorticoid receptor DNA sequence and said nucleic acid sequence for a marker protein utilizing a DNA linker sequence.

In another preferred embodiment, said glucocorticoid receptor DNA sequence encompasses the 5′ end of said sequence through and including the complete nuclear localization sequence of said glucocorticoid receptor DNA sequence.

In another preferred embodiment, said glucocorticoid receptor DNA sequence encompasses the 5′ end of said sequence through and including the complete nuclear localization sequence and the complete helix 1 sequence of said glucocorticoid receptor DNA sequence.

In another preferred embodiment, said glucocorticoid receptor DNA sequence encompasses the 5′ end of said sequence through and including DNA bases corresponding to about amino acid residue 570of said glucocorticoid receptor protein.

In another preferred embodiment, said superfamily receptor DNA sequence encompasses the 3′ end of said sequence through and including the complete ligand binding domain sequence and the complete helix 3 sequence of said superfamily receptor DNA sequence.

In another preferred embodiment, said superfamily receptor DNA sequence encompasses the 3′ end of said sequence through and including the complete ligand binding domain sequence, the complete helix 3 sequence, and at most a fragment of helix 1 of said superfamily receptor DNA sequence.

Applicants have modified the ligand binding domain (LBD) of GFP-GR in such a way that the unique cytoplasmic/nuclear translocation activity of GR is maintained, but the modified chimera responds to a superfamily receptor ligand.

In developing such a system, Applicants overcame several potential problems. First, insofar as they were then understood, the nuclear localization signals of GR were embedded within the LBD and the C-terminal region of the DNA-binding domain. Thus, complete replacement of the GR LBD might disrupt the regulated translocation signals we intended to utilize.

Second, GR interacts in the cytoplasm with a complex array of chaperon proteins, including Hsp90 and Hsp70, and ligand-dependent displacement of these proteins was thought to be intimately involved in the translocation process. Interaction domains critical for HSP90 binding are located within helix 1 of the GR LBD. Thus, removal or alteration of the GR LBD could have disrupted both the chaperone interaction domains and the nuclear localization signals in this region of the protein.

With this perspective, the inventive GFP-GR-RAR fusions were generated in which the GR LBD was replaced by the corresponding domain from RAR, preserving the GR helix 1 region intact. The chimeric molecules were then evaluated for transactivation potential and cellular distribution of the chimeric proteins. The chimeric protein behaved as an RA-dependent transactivator, but was nonresponsive to GR agonists.

The availability of crystal structures for the LBD of several receptors has led us to a compelling model for LBD structural rearrangements involved in ligand binding and coactivator interactions. In contrast, the receptor transitions responsible for intracellular trafficking have not been well understood in the art, and no general model has previously existed to account for the subcellular distribution of the steroid, nuclear, and orphan receptor superfamily.

The inventive chimeras provide a new method for elucidating mechanisms of ligand induced nuclear translocation. The ability to swap LBDs and obtain novel ligand-regulated translocation has important implications for the structural requirements necessary to maintain ligand-mediated translocation and will serve as a valuable tool in elucidating how the ligand-induced structural transition is transmitted from the LBD to domains of the chimeric protein critical for translocation.

Each member of the steroid, nuclear, and orphan receptor superfamily displays a distinct subcellular distribution, particularly in terms of the partitioning between the cytoplasmic and nuclear compartments. Some receptors, such as the glucocorticoid receptor, show primarily a cytoplasmic location in the absence of ligand, but translocate rapidly, and almost completely, to the nuclear compartment after hormone activation.

Nuclear hormone receptors form an evolutionarily related superfamily of proteins that are involved in many physiological processes and diseases. Because of this involvement, the nuclear hormone receptors are a superfamily of crucial medical significance. Many nuclear receptors bind hormones, mediating the transcriptional response to a ligand. Others, however, have no known ligand.

To date, about 70-75 different superfamily receptors, including both classical nuclear receptors with known ligands and so-called orphan receptors, which are receptors without a ligand or with an unknown ligand, have been identified. They constitute a family of transcription factors that share a modular structure of 5-6 conserved domains encoding specific functions. The most prominent distinction to other transcription factors is their capacity specifically to bind small hydrophobic molecules. These ligands constitute regulatory signals, which change the transcriptional activity of the corresponding superfamily receptor after binding. Superfamily receptors are classified by homology to other family members, with the DNA binding domain and the ligand-binding domain having the highest evolutionary conservation.

Genetic programs consist typically of several hundred genes that are expressed in a spatially and temporally controlled fashion. Superfamily receptors often act as master ‘switches’ to initiate specific genetic programs that, for example, lead to cell differentiation, proliferation or apoptosis, or regulate homeostasis. In the context of other programs these genetic activities support or initiate complex physiological phenomena, such as reproduction and organ function. Once activated by the cognate ligand, nuclear receptors regulate the primary and secondary target gene expressions that make up the corresponding physiological event. Throughout the life cycle of a multicellular organism, the coordinate interplay between programs defining cell fates in different tissues, organs and finally the entire body is at the foundation of the organism's development and subsistence.

Superfamily receptors are composed of 5-6 regions that have generally modular character. In general, the N-terminal A/B region harbors one or more autonomous transcriptional activation function (AF-1), which, when linked to a heterologous DNA binding domain, can activate transcription in a constitutive manner. When comparing superfamily receptors from different subfamilies and groups, the A/B region displays the weakest evolutionary conservation, and the distinction between the A and B regions is not always evident. A/B regions differ significantly in their length, ranging from 23 to about 550 amino acids.

The highly conserved domain C harbors the DNA-binding domain (DBD) of superfamily receptors that confers sequence-specific DNA recognition. The DNA-binding domain is mainly composed of two zinc-finger motifs. There are three prototypic DNA-binding modes of superfamily receptors: (1) a homodimer that binds to a palindromic response element, (2) an anisotropic heterodimeric complex on a DR1 direct repeat; and (3) a monomer that binds to an extended hexameric motif, the so-called NBRE.

The D region of superfamily receptors appears to correspond to a hinge between the highly structured C and E domains. It might allow the DNA and ligand-binding domains to adopt several different conformations without creating steric hindrance problems. Note in this respect that the C and the E regions contribute dimerization interfaces allowing some receptors to accommodate different heterodimerization partners and different types of response elements. Region D contains a nuclear localization signal (NLS), or at least some elements of a functional NLS. The intracellular localization of superfamily receptors is a result of a dynamic equilibrium between nuclear-cytoplasmic and cytoplasmic-nuclear shuttling. At equilibrium the large majority of receptors are nuclear, although the glucocorticoid receptor resides at cytoplasmic locations in the absence of its cognate ligand and translocates to the nucleus in a ligand-induced fashion.

A key feature of a superfamily receptor is its ligand-binding domain (LBD) in the E region. This domain is highly structured, and encodes a wealth of distinct functions, most of which operate in a ligand-dependent manner. The LBD often harbors a repression function, a major dimerization interface, and the ligand-dependent activation function AF-2. A structure-based sequence alignment of all known superfamily receptor primary amino-acid sequences strongly supports a common fold for all superfamily receptor LBDs, which has been fully confirmed when the crystal structures of multiple other NR LBDs were solved.

The general fold of nuclear receptors consists of a three-layered α-helical sandwich. Further structural features are one β-hairpin and connecting loops of variable lengths. The helices have been designated H1 to H12, starting with the most N-terminal H1, and form a hydrophobic cavity that accommodates the hydrophobic ligands. The LBD is structurally defined as the domain generated by the elements between the beginning of helix H1 and the end of helix H12. The E domain undergoes a significant conformational change upon ligand binding.

Some receptors possess at the C-terminus of the ligand binding domain a region F, which displays little evolutionary conservation. The role of the C-terminal region F is unknown. Recent literature suggests that the F region might play a role in coactivator recruitment to the E domain and in determining the specificity of the ligand binding domain coactivator interlace, perhaps by fine tuning the molecular events associated with the transcriptional properties of the E domain or the entire receptor.

As will be apparent to one of ordinary skill in the art, based on Applicants' teachings herein, some variation in the length and composition of the linker polypeptide regions is to be expected. Such minor variations, not significantly affecting the tertiary structure of the chimeric protein, are permitted in Applicants' methods of making the inventive DNA chimeras and corresponding proteins.

Further, as there are as many as six DNA codons for a single amino acid, it will also be apparent that variations in the DNA sequence which do not affect the protein sequence are inconsequential to the methods of the present invention. Such variations are to be expected in a population and are within the scope of the present inventive subject matter.

Finally, it will be apparent to one of ordinary skill in the art, based on Applicants' teachings herein, that variations in a protein sequence are also to be expected in a population, and are within the scope of the present inventive subject matter. For example, conservative substitutions are permissible in many regions affecting the general conformation, and not the binding specificity, of a protein.

### Synthesis of Compounds of the Invention

The nucleic acid chimeras and chimeric proteins of the present invention may be readily prepared by standard techniques of molecular biology, utilizing techniques known to those of ordinary skill in the art, as described in greater detail herein.

The products and intermediates may be isolated or purified using one or more standard purification techniques known to one of ordinary skill in the art, including, for example, one or more of simple solvent evaporation, recrystallization, distillation, sublimation, filtration, polymerase chain reaction, Southern blotting, Northern blotting, Western blotting, chromatography, including thin-layer chromatography, affinity chromatography, gel filtration chromatography, ion exchange chromatography, FPLC, HPLC (e.g. reverse phase HPLC), column chromatography, flash chromatography, radial chromatography, trituration, salt precipitation, two-phase separation, polymer precipitation, heat denaturation, isoelectric separation, dialysis, and the like.

### Proteins Produced by the Inventive Methods

An exemplary method for producing proteins according to the inventive method could encompass the following procedure: using conventional techniques well known in the art, a protein may be produced by the inventive methods by covalently connecting (i) a glucocorticoid receptor DNA sequence coding for the 5′ end of the DNA sequence of the glucocorticoid receptor protein, through and including the complete nuclear localization sequence and the complete helix 1 sequence of said glucocorticoid receptor DNA sequence, (ii) a superfamily receptor DNA sequence coding for the 3′ end of the DNA sequence of a superfamily receptor protein, through and including the complete ligand binding domain sequence and the complete helix 3 sequence of said superfamily receptor DNA sequence, and (iii) a nucleic acid sequence for a marker protein domain, to form a DNA chimera,

wherein said superfamily receptor DNA sequence is connected to the 3′ end of said glucocorticoid receptor DNA sequence,

wherein said marker protein domain DNA sequence is covalently connected to the 5′ end of said glucocorticoid receptor DNA sequence,

and wherein said translocation domain of the glucocorticoid receptor and said ligand binding domain of a superfamily receptor are covalently connected by a DNA linker sequence; and

expressing said DNA chimera in an expression system to prepare said protein.

It should be recognized that one skilled in the art may vary the exemplary inventive methods as set forth herein. Such variations are expected to be within the scope of the inventive subject matter.

### Nucleic Acid Chimeras

The present invention further relates to a nucleic acid chimera comprising:

a nucleic acid sequence which codes for the cytoplasmic/nuclear translocation domain of glucocorticoid receptor protein; and

a nucleic acid sequence which codes for the ligand binding domain of a superfamily receptor protein.

In a preferred embodiment, said nucleic acid chimera additionally comprises a nucleic acid sequence for a marker protein domain.

In a more preferred embodiment, said marker protein domain encodes a fluorescent protein.

In another preferred embodiment, said glucocorticoid receptor DNA sequence encompasses the 5′ end of said sequence through and including the complete nuclear localization sequence of said glucocorticoid receptor DNA sequence.

In another preferred embodiment, said glucocorticoid receptor DNA sequence encompasses the 5′ end of said sequence through and including the complete nuclear localization sequence and the complete helix 1 sequence of said glucocorticoid receptor DNA sequence.

In another preferred embodiment, said glucocorticoid receptor DNA sequence encompasses the 5′ end of said sequence through and including DNA bases corresponding to about amino acid residue 570of said glucocorticoid receptor protein.

In another preferred embodiment, said superfamily receptor DNA sequence encompasses the 3′ end of said sequence through and including the complete ligand binding domain sequence and the complete helix 3 sequence of said superfamily receptor DNA sequence.

In another preferred embodiment, said superfamily receptor DNA sequence encompasses the 3′ end of said sequence through and including the complete ligand binding domain sequence, the complete helix 3 sequence, and at most a fragment of helix 1 of said superfamily receptor DNA sequence.

In another preferred embodiment, said ligand binding domain is the ligand binding domain of estrogen receptor.

In a particularly preferred embodiment, said nucleic acid chimera is SEQ. ID NO. 1.

In another preferred embodiment, said ligand binding domain is the ligand binding domain of retinoic acid receptor.

In a particularly preferred embodiment, said nucleic acid chimera is SEQ. ID NO. 2.

### Chimeric Proteins

The present invention further relates to chimeric protein comprising two elements:

a glucocorticoid receptor 5′ end, encompassing the nuclear translocation domain and helix 1; and

a superfamily receptor 3′ end, encompassing the ligand binding domain and helix 3.

In a preferred embodiment, said chimeric protein further comprises a marker protein domain.

In a particularly preferred embodiment, said marker protein domain encodes a fluorescent protein.

In another preferred embodiment, said ligand binding domain is the ligand binding domain of estrogen receptor.

In a particularly preferred embodiment, said chimeric protein is SEQ. ID NO. 3.

In another preferred embodiment, said ligand binding domain is the ligand binding domain of retinoic acid receptor.

In a particularly preferred embodiment, said chimeric protein is SEQ. ID NO. 4.

As discussed above, chimeric proteins of the present invention are produced using conventional techniques well known in the art.

### Methods for Detecting a Ligand

The present invention further relates to a method for detecting a ligand of a superfamily receptor protein, which comprises:

producing a nucleic acid vector encoding a nucleic acid chimera comprising three elements: a 5′ end of a glucocorticoid receptor, encompassing the nuclear translocation domain and helix 1, a 3′ end of a superfamily receptor, encompassing the ligand binding domain and helix 3, and a nucleic acid sequence for a marker protein domain;

transfecting a eukaryotic cell with said nucleic acid vector;

isolating a clonal population of cells that express a chimeric protein translated from said nucleic acid vector;

contacting said cells with a sample compound or composition; and

detecting the presence of cytoplasmic/nuclear translocation in response to a ligand of said ligand binding domain.

In a preferred embodiment, said marker protein domain encodes a fluorescent protein.

### Methods for Determining Concentration of a Ligand

The present invention further relates to a method for determining the concentration of a ligand of a labeled chimeric superfamily receptor protein, which comprises:

producing a nucleic acid vector encoding a nucleic acid chimera comprising: a nucleic acid sequence coding for the cytoplasmic/nuclear translocation domain of glucocorticoid receptor protein, a nucleic acid sequence coding for the ligand binding domain of a superfamily receptor protein, and a nucleic acid sequence for a marker protein domain;

transfecting a eukaryotic cell with said nucleic acid vector;

isolating a clonal population of transfected cells that express a chimeric protein translated from said nucleic acid vector;

contacting said transfected cells with a sample;

scanning one or more test cell(s) to obtain signal data from said labeled protein;

converting said signal data to obtain the cellular location of said labeled protein in said test cell(s); and

analyzing said data using an analysis system having an algorithm to calculate changes in the distribution of said labeled protein between the cell cytoplasm and the cell nucleus of said test cell(s), said analysis system having the capability of providing an accurate reading of the concentration of a ligand.

In a preferred embodiment, said marker protein domain encodes a fluorescent protein.

It will be apparent to one of ordinary skill in the art that there are a number of commercially available detection and analysis systems which may be interchangeably incorporated into the inventive methods. Such variations are contemplated to be within the scope of the inventive subject matter.

### Kits for Screening an Environmental Sample

In addition, the present invention relates to a kit for detecting and screening for a ligand of a superfamily receptor protein in an environmental sample, comprising:

a cell-based system which expresses a chimeric protein comprising the cytoplasmic/nuclear translocation domain of glucocorticoid receptor protein, the ligand binding domain of a superfamily receptor protein, and a marker protein domain; and

a detection system for the detection of the translocation of said marker protein.

In a preferred embodiment, said kit additionally comprises one or more compounds and/or compositions which stably associate with said chimeric protein in the absence of a ligand for the ligand binding domain of said chimeric protein, and which dissociates from said chimeric protein in the presence of a ligand for the ligand binding domain of said chimeric protein.

### Cell-free Kits for Screening an Environmental Sample

In addition, the present invention relates to a kit for detecting and screening for a ligand of a superfamily receptor protein in an environmental sample, comprising:

a quantity of a chimeric protein comprising the cytoplasmic/nuclear translocation domain of glucocorticoid receptor protein, the ligand binding domain of a superfamily receptor protein, and a marker protein domain;

a cell-free membrane system which restricts translocation of the chimeric protein when no ligand is bound to the ligand binding domain of said chimeric protein, and which permits translocation of the chimeric protein when the ligand binding domain of said chimeric protein is bound to its ligand; and

a detection system for the detection of the translocation of said marker protein.

In a preferred embodiment, said kit additionally comprises one or more compounds and/or compositions which stably associate with said chimeric protein in the absence of a ligand for the ligand binding domain of said chimeric protein, and which dissociates from said chimeric protein in the presence of a ligand for the ligand binding domain of said chimeric protein.

### Methods for Diagnosis of Nuclear Transport Defects

Translocation across the nuclear membrane involves a ligand-bound receptor, having at least one nuclear or cytoplasmic localization sequence, importin or exportin proteins, and the protein complex comprising the nuclear pore. For translocation to the nucleus from the cytoplasm, a ligand-bound receptor is recognized by an importin, which in turn is recognized by nuclear pore protein(s) and transported across the nuclear membrane. However, defects in this process are not readily distinguishable from other defects or impediments in the gene expression cascade, including, for example, improper ligand or receptor expression or folding; defective chaperoning processes; improper second messenger protein expression or folding; and defective transcription processes, to name but a few; all of these defects result in the same phenotypic defect: lack of gene expression. It would be very valuable to be able to identify, or exclude, nuclear transport defects from the long list of possible defects in the gene expression process.

The present invention thus relates to a method for diagnosis of defects in the nuclear transportation process, which comprises:

producing a nucleic acid vector encoding a nucleic acid chimera comprising: a nucleic acid sequence coding for the cytoplasmic/nuclear translocation domain of glucocorticoid receptor protein, a nucleic acid sequence coding for the ligand binding domain of a superfamily receptor protein, and a nucleic acid sequence for a marker protein domain;

transfecting a set of suspected defective cells with said nucleic acid vector;

isolating a clonal population of said cells that express a chimeric protein translated from said nucleic acid vector;

contacting said cells with a ligand of said ligand binding domain; and

detecting the presence or absence of cytoplasmic/nuclear translocation in response to said ligand.

### Methods for Treating Translocation Defect Disorders

Finally, the present invention relates to a method for treating defective translocation of a superfamily receptor protein from the cytoplasm to the nucleus of a cell, in an animal in need thereof, comprising:

producing a nucleic acid vector which is capable of being transcribed, and which encodes a nucleic acid chimera comprising: a nucleic acid sequence coding for the cytoplasmic/nuclear translocation domain of glucocorticoid receptor protein and a nucleic acid sequence coding for the ligand binding domain of said superfamily receptor protein;

transfecting a target cell in said animal with said nucleic acid vector.

A vector of the present invention capable of being transcribed may comprise an effective amount of a recombinant nucleic acid sequence or the protein product of such a recombinant nucleic acid sequence, in combination with a pharmaceutically acceptable carrier. The effective amount of a recombinant protein will generally comprise from about 0.1 mg to about 100 mg of the active agent per kilogram of patient body weight per day. The effective amount can vary depending upon the physical status of the patient and other factors well known in the art. Moreover, it will be understood that this dosage of active agent can be administered in a single or multiple dosage units to provide the desired therapeutic effect. If desired, other therapeutic agents can be employed in conjunction with those provided by the present invention.

The compositions of the invention are preferably delivered to the patient by means of a pharmaceutically acceptable carrier. Such carriers are well known in the art and generally will be in either solid or liquid form. Solid form pharmaceutical preparations which may be prepared according to the present invention include powders, tablets, dispersible granules, capsules, cachets and suppositories. In general, solid form preparations will comprise from about 5% to about 90% by weight of the active agent.

The compositions of the invention may be provided in any suitable dosage form known in the art. For example, the compositions may be incorporated into tablets, powders, granules, beads, chewable lozenges, capsules, liquids, or similar conventional dosage forms, using conventional equipment and techniques know in the art.

Further, the dosage form can be in the form of a bi-layer tablet composed of at least one extended-release layer and at least one immediate-release layer. Also, the bi-layer tablet can be coated for ease of administration or can be enteric coated to reduce any gastric irritation and the unpleasant “burping” produced by the vitamins and minerals. Also, multi-particulate design of extended-release and immediate-release components can be enteric coated and compressed into a tablet or filled into hard or soft gelatin capsules.

When preparing dosage forms incorporating the compositions of the invention, the compositions are normally blended with conventional excipients such as binders, including gelatin, pregelatinized starch, and the like; lubricants, such as hydrogenated vegetable oil, stearic acid, and the like; diluents, such as lactose, mannose, and sucrose; disintegrants, such as carboxymethyl cellulose and sodium starch glycolate; suspending agents, such as povidone, polyvinyl alcohol and the like; absorbent, such as silicon dioxide; preservatives, such as methylparaben, propylparaben, and sodium benzoate; surfactants, such as sodium lauryl sulfate, polysorbate 80, and the like; and colorants, such as F.D. & C dyes.

For preparing compositions from the compounds described by this invention, inert, pharmaceutically acceptable carriers can be used which are either solid or liquid. Solid form preparations include powders, tablets, dispersible granules, capsules, and cachets. A solid carrier can be one or more substances which may also act as diluents, flavoring agents, solubilizers, lubricants, suspending agents, binders or tablet disintegrating agents; it can also be encapsulating material. In powders, the carrier is a finely divided solid which is in admixture with the finely divided active compounds. In the tablet the active compound is mixed with carrier having the necessary binding properties in suitable proportions and compacted in the shape and size desired. The powders and tablets preferably contain from 5 or 10 to about 90 percent of the active ingredient. Suitable solid carriers are magnesium carbonate, magnesium stearate, talc, sugar, lactose, pectin, dextrin, starch, gelatin, tragacanth, methylcellulose, sodium carboxymethylcellulose, a low melting wax, coca butter, and the like. The term “preparation” is intended to include the formulation of the active compounds with encapsulating material as carrier providing a capsule in which the active component (with or without other carriers) is surrounded by carrier, which is thus in association with it. Similarly, cachets are included. Tablets, powders, cachets, and capsules can be used a solid dosage forms suitable for oral administration. Liquid form preparations include solutions, suspensions, and emulsions. As an example, water or water/propylene glycol solutions for parenteral injection may be used. Liquid preparations can also be formulated in solution in aqueous polyethylene glycol solution. Aqueous solutions suitable for oral use can be prepared by dissolving the active component in water and adding suitable colorants, flavors, stabilizing and thickening agents as desired. Aqueous suspensions suitable for oral use can be made by dispersing the finely divided active component in water with viscous material, i.e., natural or synthetic gums, resins, methylcellulose, sodium carboxymethylcellulose, and other well-known suspending agents.

Also included are solid form preparations which are intended to be converted, shortly before use, to liquid form preparations for either oral or parenteral administration. Such liquid forms include solutions, suspensions, and emulsions. These particular solid form preparations are most conveniently provided in unit dose form and as such are used to provide a single liquid dosage unit. Alternately, sufficient solid may be provided so that after conversion to liquid form, multiple individual liquid doses may be obtained by measuring predetermined volumes of the liquid form preparation as with a syringe, teaspoon, or other volumetric container. When multiple liquid doses are so prepared, it is preferred to maintain the unused portion of said liquid doses at low temperature, i.e., under refrigeration, in order to retard possible decomposition.

The solid and liquid forms may contain, in addition to the active material, flavorants, colorants, stabilizers, buffers, artificial and natural sweeteners, dispersants, thickeners, solubilizing agents, and the like. The liquid utilized for preparing the liquid form preparation may be water, isotonic water, ethanol, glycerine, propylene glycol, and the like as well as mixtures thereof.

Preferably, the preparations are in unit dosage form. In such form, the preparation is subdivided into unit doses containing appropriate quantities of the active components. The unit dosage form can be a packaged preparation, the package containing discrete quantities of preparation, for example, packeted tablets, capsules, and powders in vials or ampoules. The unit dosage form can also be a capsule, cachet, or tablet itself or it can be the appropriate number of any of these in packaged form.

The quantity of active compound in a unit dose of preparation may be varied according to the particular application and the potency of the active ingredients. Determination of the proper dosage for a particular situation is within the skill of the art. For convenience, the total daily dosage may be divided and administered in portions during the day if desired or at one time, morning, afternoon, night as well as biphasic, triphasic, etc. Controlled and uncontrolled release formulations are also contemplated.

Although the products of the invention are preferably intended for administration to humans, it will be understood that the formulation may also be utilized in veterinary therapies for other animals.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby to solidify.

The pharmaceutical preparations of the invention may include one or more preservatives well known in the art, such as benzoic acid, sorbic acid, methylparaben, propylparaben and ethylenediaminetetraacetic acid (EDTA). Preservatives are generally present in amounts up to about 1% and preferably from about 0.05 to about 0.5% by weight of the pharmaceutical composition.

Useful buffers for purposes of the invention include citric acid-sodium citrate, phosphoric acid-sodium phosphate, and acetic acid-sodium acetate in amounts up to about 1% and preferably from about 0.05 to about 0.5% by weight of the pharmaceutical composition. Useful suspending agents or thickeners include cellulosics like methylcellulose, carageenans like alginic acid and its derivatives, xanthan gums, gelatin, acacia, and microcrystalline cellulose in amounts up to about 20% and preferably from about 1% to about 15% by weight of the pharmaceutical composition.

Sweeteners which may be employed include those sweeteners, both natural and artificial, well known in the art. Sweetening agents such as monosaccharides, disaccharides and polysaccharides such as xylose, ribose, glucose, mannose, galactose, fructose, dextrose, sucrose, maltose, partially hydrolyzed starch or corn syrup solids and sugar alcohols such as sorbitol, xylitol, mannitol and mixtures thereof may be utilized in amounts from about 10% to about 60% and preferably from about 20% to about 50% by weight of the pharmaceutical composition. Water soluble artificial sweeteners such as saccharin and saccharin salts such as sodium or calcium, cyclamate salts, acesulfame-K, aspartame and the like and mixtures thereof may be utilized in amounts from about 0.001% to about 5% by weight of the composition.

Flavorants which may be employed in the pharmaceutical products of the invention include both natural and artificial flavors, and mints such as peppermint, menthol, vanilla, artificial vanilla, chocolate, artificial chocolate, cinnamon, various fruit flavors, both individually and mixed, in amounts from about 0.5% to about 5% by weight of the pharmaceutical composition.

Colorants useful in the present invention include pigments which may be incorporated in amounts of up to about 6% by weight of the composition. A preferred pigment, titanium dioxide, may be incorporated in amounts up to about 1%. Also, the colorants may include other dyes suitable for food, drug and cosmetic applications, known as F.D.&C. dyes and the like. Such dyes are generally present in amounts up to about 0.25% and preferably from about 0.05% to about 0.2% by weight of the pharmaceutical composition. A full recitation of all F.D.&C. and D.&C. dyes and their corresponding chemical structures may be found in the Kirk-Othmer Encyclopedia of Chemical Technology, in Volume 5, at pages 857-884, which text is accordingly incorporated herein by reference.

Useful solubilizers include alcohol, propylene glycol, polyethylene glycol and the like and may be used to solubilize the flavors. Solubilizing agents are generally present in amounts up to about 10%; preferably from about 2% to about 5% by weight of the pharmaceutical composition.

Lubricating agents which may be used when desired in the instant compositions include silicone oils or fluids such as substituted and unsubstituted polysiloxanes, e.g., dimethyl polysiloxane, also known as dimethicone. Other well known lubricating agents may be employed.

It is not expected that compounds of the present invention will display significant adverse interactions with other synthetic or naturally occurring substances. Thus, a compound of the present invention may be administered in combination with other compounds and compositions. In particular the compounds of the present invention may be administered in combination with other compounds of the present invention and other targeted nuclear transcription elements substances.

The optimal pharmaceutical formulations will be determined by one skilled in the art depending upon considerations such as the route of administration and desired dosage. Such formulations may influence the physical state, stability, rate of in vivo release, and rate of in vivo clearance of the present therapeutic agents of the invention.

### Route(s) of Administration

The route(s) of administration of the compounds and compositions of the present invention are well known to those skilled in the art (see, for example, “Remington's Pharmaceutical Sciences”, 18th Edition, Chapter 86, pp. 1581-1592, Mack Publishing Company, 1990). The compounds and compositions may be administered orally, parenterally, by inhalation spray, topically, rectally, nasally, buccally, vaginally, or via an implanted reservoir in dosage formulations containing conventional non-toxic pharmaceutically-acceptable carriers, adjuvants, and vehicles. The term parenteral as used herein includes subcutaneous, intravenous, intramuscular, intraperitoneally, intrathecally, intraventricularly, intrasternal, and intracranial injection or infusion techniques.

The compounds and compositions may be administered in the form of sterile injectable preparations, for example, as sterile injectable aqueous or oleaginous suspensions. These suspensions, may be formulated according to techniques known in the art using suitable dispersing or wetting agents and suspending agents. The sterile injectable preparations may also be sterile injectable solutions or suspensions in non-toxic parenterally-acceptable diluents or solvents, for example, as solutions in 1,3-butanediol. Among the acceptable vehicles and solvents that may be employed are water, Ringer's solution and isotonic sodium chloride solution. In addition, sterile, fixed oils are conventionally employed as solvents or suspending mediums. For this purpose, any bland fixed oil such as a synthetic mono- or di-glyceride may be employed. Fatty acids such as oleic acid and its glyceride derivatives, including olive oil and castor oil, especially in their polyoxyethylated versions, are useful in the preparation of injectables. These oil solutions or suspensions may also contain long-chain alcohol diluents or dispersants.

Additionally, the compounds and compositions may be administered orally in the form of capsules, tablets, aqueous suspensions, or solutions. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. The oral dosage forms may further contain sweetening, flavoring, coloring agents, or combinations thereof. Delivery in an enterically coated tablet, caplet, or capsule, to further enhance stability and provide release in the intestinal tract to improve absorption, is the best mode of administration currently contemplated.

The compounds may also be administered rectally in the form of suppositories. These compositions can be prepared by mixing the drug with a suitable non-irritating excipient which is solid at room temperature, but liquid at rectal temperature and, therefore, will melt in the rectum to release the drug. Such materials include cocoa butter, beeswax, and polyethylene glycols.

Furthermore, the compounds may be administered topically, especially when the conditions addressed for treatment involve areas or organs readily accessible by topical application, including the lower intestinal tract. Suitable topical formulations can be readily prepared for such areas or organs. For example, topical application to the lower intestinal tract can be effected in a rectal suppository formulations (see above) or in suitable enema formulations.

It is envisioned that the continuous administration or sustained delivery of the compounds and compositions of the present invention may be advantageous for a given condition. While continuous administration may be accomplished via a mechanical means, such as with an infusion pump, it is contemplated that other modes of continuous or near continuous administration may be practiced. For example, such administration may be by subcutaneous or muscular injections as well as oral pills.

Techniques for formulating a variety of other sustained- or controlled-delivery means, such as liposome carriers, bio-erodible particles or beads and depot injections, are also known to those skilled in the art.

### Dosage

Dosage levels of chimeric proteins and nucleic acid chimeras are known to those of ordinary skill in the art. The amount of active ingredient that may be combined with the carrier materials to produce a single dosage form will vary depending upon the host treated and the particular mode of administration.

It is understood, however, that a specific dose level for any particular patient will depend upon a variety of factors, including the activity of the specific compound employed; the age, body weight, general health, sex and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

### EXAMPLES

The following examples are illustrative of the present invention and are not intended to be limitations thereon. Unless otherwise indicated, all percentages are based upon 100% by weight of the final composition.

### Example 1

Production of a GFP-GR-RAR Chimera

The 1471.1 cell line was used for this transfection study. Cells were maintained in DMEM with 10% characterized fetal calf serum, L-glutamine, and antibiotics. For transient transfections, 2×10⁵ cells were resuspended in cold DMEM without serum with the indicated plasmids and incubated on ice for 1 minute. Electroporations were done in an electroporator at 140 V, 3 pulses and 10 msec pulse length followed by a 10 minute recovery on ice. Cells were then plated and allowed to recover overnight in complete media.

Receptor Chimera. Sequences 3′ to the coding region for helix 1 of the LBD of GFP-tagged rat GR were replaced by a multiple cloning site polylinker as follows: a PCR fragment was generated to contain GR sequences including a unique Sph I site and extending through the end of the LBD H1 with the polylinker fused at the 3′ end, using suitable 5′ and 3′ primers. The PCR product was restricted with Sph I and Xba I and inserted at the corresponding Sph I and Xba I sites in pCI-nGFP-C656G. A version tagged with EGFP was also made by cloning the Nhe I-filled in BspE I fragment from EGFP-C1 between the Nhe I and filled in BssH II sites of the LBD-deleted C656G GR. The LBD-deleted C656G GR constructs were confirmed by sequencing.

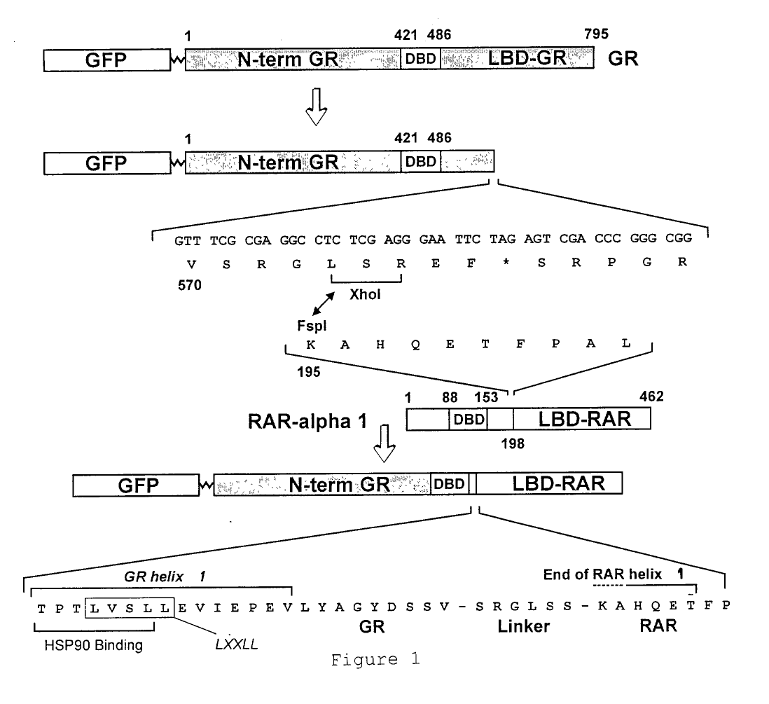

Different GR-RAR chimeras were generated by cloning portions of the LBD from a human RAR-alpha cDNA in-frame into the multiple cloning site polylinker of LBD-deleted GR. The chimera shown in FIG. 1 was made by fusing the RAR-LBD, starting at the end of its H1 coding region, from amino acid residue 195, and extending through the end of coding sequence and 300 bases of 3′ untranslated region, to the end of the GR LBD helix 1 region. The RAR-LBD-containing fragment from filled in Fsp I to Eco RI was cloned between the filled in Xho I and the Eco RI sites of LBD-deleted C656G GR, and was confirmed by sequencing.

Fluorescence Microscopy. For fluorescence microscopy, cells were electroporated with 2 ug GFP-GR-RAR, GFP-GR, or GFP-RAR. After electroporation, 1/24th of the cells were plated in each well of a 6-well dish onto a clean #1.5 glass coverslip in DMEM containing 10% charcoal/dextran-treated FCS. The next day, cells were treated with the indicated ligand for 1 hour, cells on coverslips were rinsed with PBS, inverted onto microscope slides, and immediately viewed on a scanning confocal microscope. GFP was excited with a 488 nm laser lines supplied from a air cooled argon laser. GFP emission was monitored between 505 and 590 nm with a pinhole setting of 1.0.

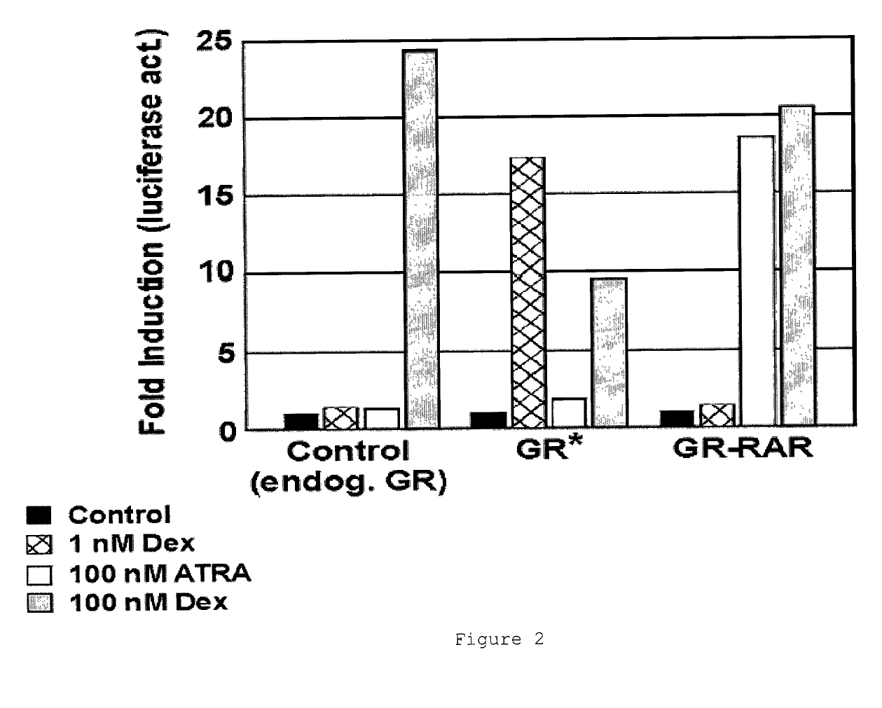

Assay of Receptor Activity. Cells were electroporated with 5 ug pLTRLuc, containing the MMTV LTR, and 0.7 ug pCMV-betaGal with or without 10 ug of the indicated receptor expression vector as described above. Cells were plated in duplicate in 100 mm dishes in DMEM containing 10% charcoal/dextran-treated FCS, as described above, and allowed to recover overnight. The next day, cells were treated with the indicated ligand for 6 hours. As shown in FIG. 2, luciferase and beta-galactosidase were assayed.

Co-immunoprecipitation Assays. Transfected cells were harvested at 36-48 hrs and lysed in 300 ul of HEGM+0.5% NP40+2 mM DTT per 107 cells for 5 min at 40° C., where HEGM is 20 mM Hepes pH7, 1 mM EDTA, 10% glycerol, 10 mM Sodium Molybdate. A protease inhibitor cocktail was also included. Nuclei were pelleted and the cytoplasmic extract was precleared with blank protein G sepharose for 2 hrs at 40° C. and then immunoadsorbed against protein G sepharose pre-loaded with anti-GFP monoclonal antibody overnight at 40° C. Sepharose pellets were then washed with 3×1 ml of HEM+50 mM NaCl+0.5% Tween 20, boiled in electrophoresis loading buffer, run on 4-12% Bis-Tris gradient gels and blotted. Western blots were sequentially probed first with anti-HSP90 monoclonal antibody, then with anti-GFP antibody, and finally with anti-GR antipeptide polyclonal antibody.

Results. Applicants have modified the ligand binding domain (LBD) of GFP-GR in such a way that the unique cytoplasmic/nuclear translocation activity of GR was maintained, but the modified chimera responded to a superfamily receptor ligand.

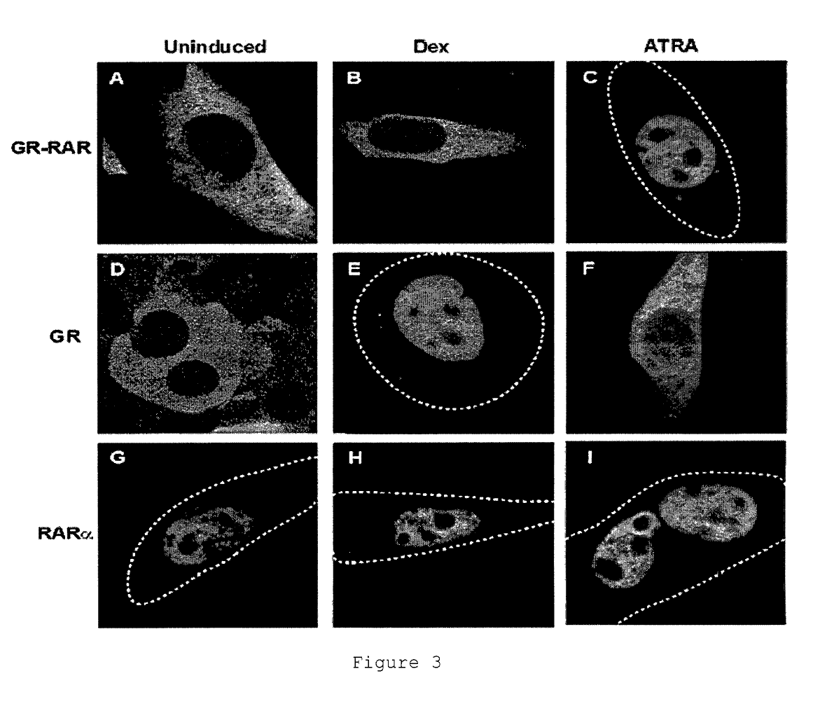

Subcellular trafficking was examined in response to ligands. Representative results are shown in FIG. 3; in each case, essentially all of the transfected cells in culture responded uniformly in the same manner as the examples shown. The GR receptor was completely cytoplasmic in the absence of ligand and became exclusively nuclear in response to dexamethasone, as shown in FIG. 3, panels D and E but was unaffected by ATRA as shown in FIG. 3, panel F. As expected, wild type RAR was always nuclear, as shown in FIG. 3, panels G-I. GR-RAR was cytoplasmic in non-treated cells, remained cytoplasmic after dexamethasone treatment, as shown in FIG. 3, panels A and B, but translocated to the nucleus with ATRA, as shown in FIG. 3, panel C. That is, the GR-RAR chimera retained the cytoplasmic localization of ligand-free wild-type GR, but translocated in response to the normal RAR ligand rather than GR agonists.

We believed that HSP90 binding by GR is important to a properly folded protein structure. Accordingly, the ability of the chimeric GR-RAR receptor to interact with HSP90 was preserved with the new LBD.

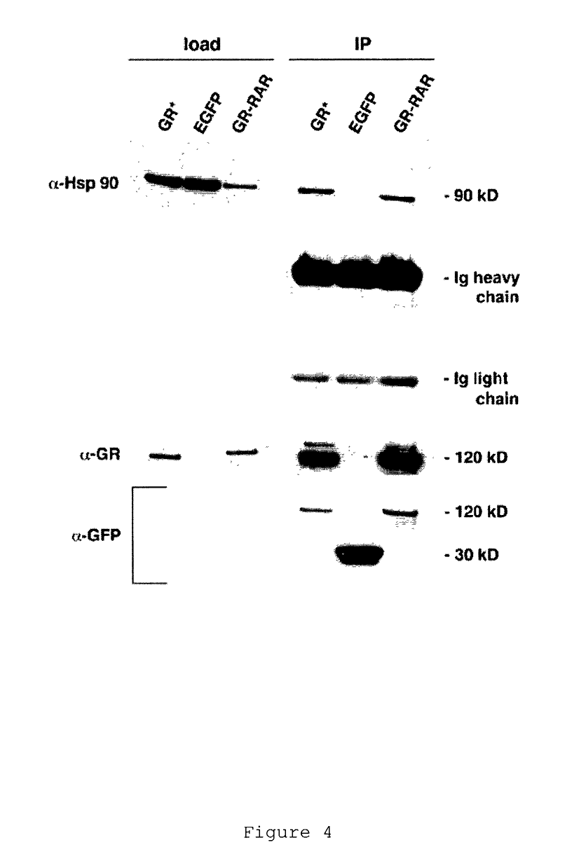

Since we also believed that the cytoplasmic interaction with HSP90 was critical for steroid binding and regulated translocation of the receptor, it was of interest to determine whether the ability of the chimeric GR-RAR receptor to interact with HSP90 was retained. Cytoplasmic extracts from cells transfected with GR, the GR-RAR chimera fused to GFP, or with native EGFP, were each immunoprecipitated with anti-GFP antibody and the precipitates were probed for the presence of bound HSP90, as shown in FIG. 4. Both cytoplasmic GR and GR-RAR chimeras interacted specifically with HSP90, while EGFP did not, demonstrating that HSP90 interactions key to ligand-regulated translocation were conserved in the chimeric protein.

FIG. 1 is a drawing which depicts the construction of a chimeric GFP-GR-RAR receptor. A GFP-tagged version of rat GR was modified with the insertion of a polylinker at amino acid position 570, replacing the LBD. A C-terminal fragment of the RAR receptor beginning at amino acid position 195 was fused to GFP-GR at an Xho I site in the polylinker. The resulting fusion protein includes the last six amino acids of the RAR LBD helix 1, as well as the complete GR LBD helix 1. Another GR domain included in the fusion is the domain required for HSP90 binding.

FIG. 2 is a graph which depicts transcriptional transactivation by the chimeric receptor. Transfected cells were treated with the indicated ligand for 6 hours. Fold inductions are shown in the bottom table, and were calculated from the luciferase activity, normalized to beta-galactosidase activity, of ligand induced samples compared to untreated controls. Experiments were carried out twice in duplicate and a representative example is shown for mock transfected cells control cells transfected with a GFP-GR C656G mutant “super” receptor GR*, and cells transfected with the GR-RAR chimeras. The data in Table 1 shows that the chimeric GR-RAR protein of the present invention is induced by all-trans retinoic acid, but not physiologically relevant concentrations of dexamethasone, and the chimeric protein translocated in the presence of its LBD ligand, ATRA.

TABLE 1
Transcriptional Activation of Control
and Chimeric Proteins

| | Control | | |
|---|---|---|---|
| | (Endogenous GR) | GR* | GR-RAR |
| Control | 1.00 | 1.00 | 1.00 |
| 1 nM Dex | 1.35 | 17.4 | 1.33 |
| 100 nM ATRA | 1.27 | 1.78 | 18.6 |

FIG. 3 is a series of photographs which depict subcellular trafficking of chimeric receptor. Mouse mammary adenocarcinoma cells were transfected with GFP-GR-RAR (panels A, B, C), GFP-GR (panels D, E, F), or GFP-RAR (panels G, H, I). Cells were treated for 1 hour with medium containing only charcoal stripped-serum (panels A, D, G), or 100 nM dexamethasone (panels B, E, H), or 100 nM all-trans-retinoic acid (panels C, F, I). The cell outline is indicated by a dashed white line in panels C, E, G, H, and I, the examples in which the subcellular distribution is nuclear. The cells shown in panels D and I are binucleate.

FIG. 4 is a photograph which depicts the interaction of the chimeric receptor with heat shock protein 90. Cytoplasmic extracts from cells transfected with GFP fusions to GR* or to GR-RAR, or with native EGFP, were immunoprecipitated using an anti-GFP monoclonal antibody, separated on a 4-12% gradient gel, blotted, and probed with anti-HSP90 antibody. About 0.5% of each extract used prior to immunoprecipitation was also run for comparison. Blots were subsequently reprobed with anti-GFP and with anti-GR antibodies as a control to confirm uniformity in binding and recovery of immunoprecipitate. The anti-GFP antibody used for western blots reacted poorly with the GFP-fusion proteins compared to unmodified EGFP and was undetectable in the load. Anti-GR was used as a second control for binding and recovery. FIG. 4 shows that the GFP-GR-RAR chimeric protein binds to HSP90 with similar affinity to GR-HSP90 binding.

For additional discussion of the procedures followed and results obtained in Example 1, Applicants' publication, Mackem S, Baumann C T, Hager G L, A glucocorticoid/retinoic acid receptor chimera that displays cytoplasmic/nuclear translocation in response to retinoic acid. A real time sensing assay for nuclear receptor ligands, J Biol Chem. 276(49):45501-4 (2001), is incorporated by reference in its entirety.

### Example 2

Production of GFP-GR-ER Chimera

Using techniques substantially similar to those described above in Example 1, Applicants have produced a GFP-GR-ER DNA chimera, which translates to a GFP-GR-ER chimeric protein which exhibits the following characteristics: (1) within the limits of detection, complete isolation of the chimeric protein in the cytoplasm of a cell in the absence of ligand, estrogen, for its LBD, and rapid, essentially complete translocation to the nucleus in the presence of estrogen at a concentration of 100 nM.

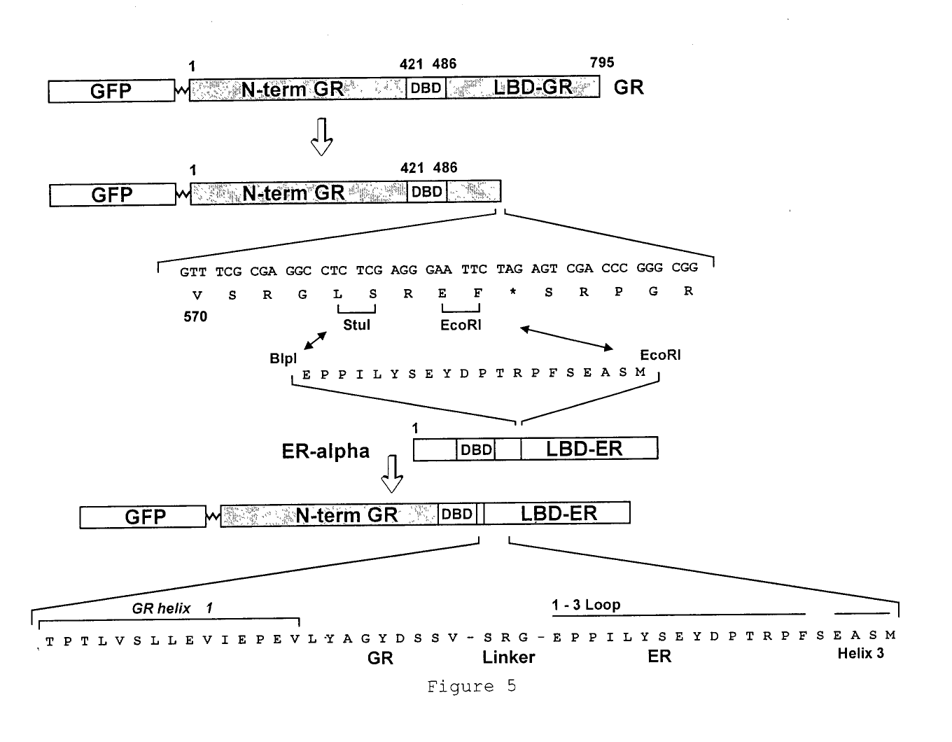

Thus, FIG. 5 depicts the construction of a chimeric GFP-GR-ER receptor. A GFP-tagged version of rat GR was modified with the insertion of a polylinker at amino acid position 570, replacing the LBD. A C-terminal fragment of a GFP-tagged ER receptor, beginning at amino acid position 323 as correlated to the ER sequence published as Accession No. NM000125, was fused to GFP-GR at a Stu I/Blp I site in the polylinker. The resulting fusion protein includes part of the ER LBD 1-3 loop as a spacer, as well as the complete GR LBD helix 1. Another GR domain included in the chimera is the heptapeptide element essential for HSP90 binding.

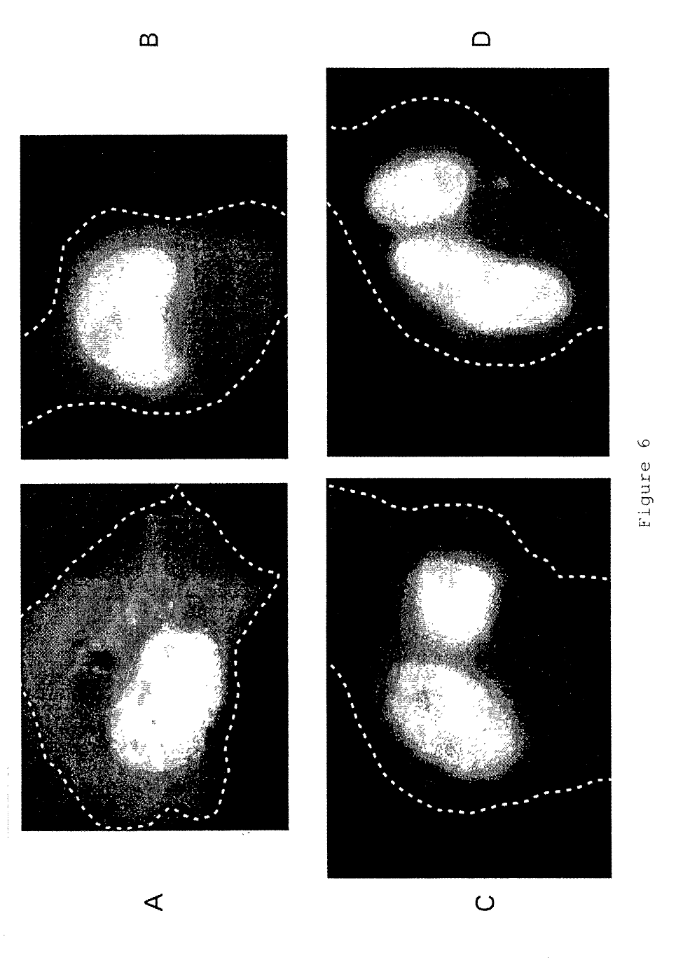

FIG. 6 is a series of photographs which depict subcellular trafficking of wild type ER receptor, without estrogen (two panels A and B) and in the presence of estrogen at 100 nM concentration (panels C and D). The cell outline is indicated by a dashed white line in all panels. As is clearly shown, wild type ER is distributed throughout the cell nucleus with or without ligand.

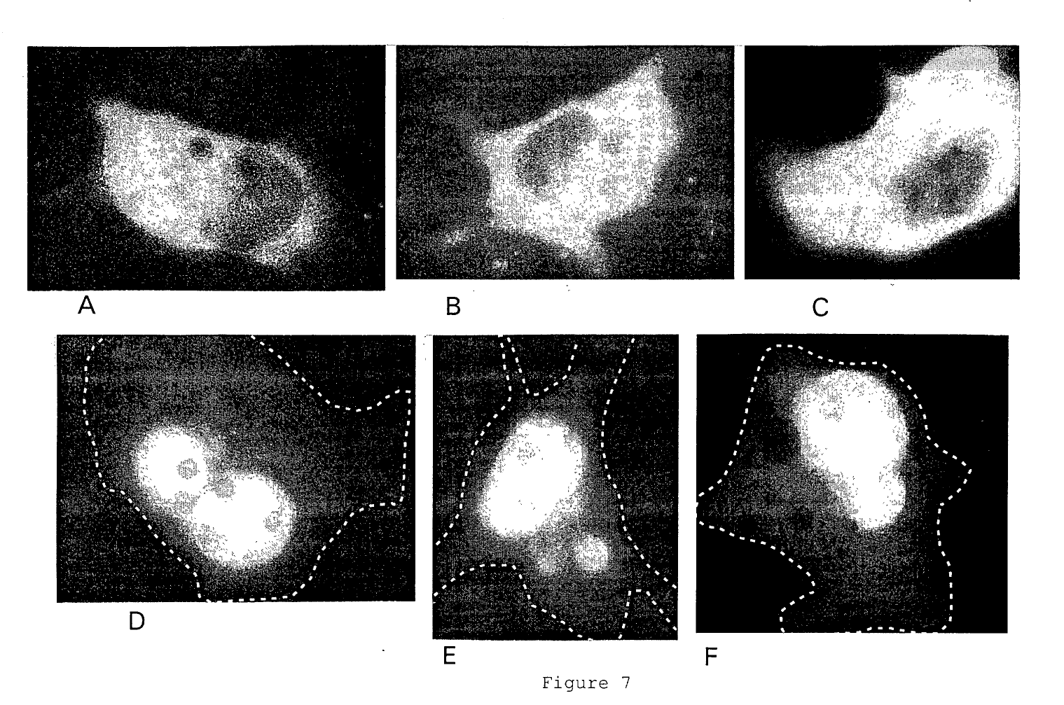

FIG. 7 is a series of photographs which depict subcellular trafficking of a chimeric GFP-GR-ER receptor, without estrogen (panels A, B, and C) and in the presence of estrogen at 100 nM concentration (panels D, E, and F). The cell outline is indicated by a dashed white line in panels D, E, and F. The cells shown in panel D is binucleate. As is clearly shown, chimeric GFP-GR-ER receptor is distributed exclusively in the cell cytoplasm without ligand, and translocates to exclusive distribution in the nucleus in the presence of ligand.

### Example 3

### Production of Additional GFP-GR-Receptor Chimeras

Almost all superfamily receptors share a highly conserved zinc-finger DNA-binding domain (DBD) and a less conserved ligand-binding domain (LBD). Pre-genome methods previously identified 48 human nuclear receptor genes. A recent study of the sequence of the human genome found all 48 known nuclear receptors, including 48 LBDs, and estimates that there are about 75 superfamily receptors. Table 2 is a catalogue of the known genes coding for superfamily receptor proteins.

TABLE 2
Candidate Receptor Proteins for GR Chimeras

| | | GenBank | |
|---|---|---|---|
| Gene | | accession | |
| nomenclature | Localization | number | Domain(s) identified |
| TRα NR1A1 | chr. 17 | AC068669 | DBD + LBD |
| TRβ NR1A2 | chr. 3 | AC018451.11 | DBD + LBD |
| RARα NR1B1 | chr. 17 | AC018629 | DBD + LBD |
| RARβ NR1B2 | chr. 3 | AC011323 | DBD |
| | chr. 3.332 | AC012037.11 | LBD |
| RARγ NR1B3 | chr. 12 | AC073573 | DBD + LBD |
| PPARα | chr. 22.431 | AL078611.1 | DBD + begining-LBD |
| | chr. 22.432 | AL078611.1 | end-LBD |
| PPARβ NR1C2 | chr. 6 | AL022721 | DBD + LBD |
| PPARγ NR1C3 | chr. 3.184 | AC016333.5 | DBD + LBD |
| Rev-erbα NR1D1 | chr. 17 | AC068669 | DBD + LBD |
| Rev-erbβ NR1D2 | chr. 3.317 | AC046128.6 | LBD |
| orphan rev-erb | chr. 2.1230 | AC012444.4 | mid-LBD |
| fragment | | | |
| RORα NR1F1 | chr. 15.575 | AC012344 | DBD + LBD |
| RORβ NR1F2 | chr. 9 | AL137018 | DBD + LBD |
| RORγ NR1F3 | chr. 1.1727 | AC068971.2 | DBD + LBD |
| LXRα NR1H2 | chr. 11.503 | AC018410 | DBD + LBD |
| LXRβ NR1H3 | chr. 19 | AC073646 | DBD + LBD |
| FXR NR1H4 | chr. 12.1078 | AC010200.7 | DBD |
| | chr. 12.1079 | AC010200.7 | end-DBD + LBD |
| FXRβ NR1H5 | chr. 1 | AL390235 | DBD + partial-LBD |
| | chr. 1 | AL138783 | LBD |
| VDR NR1I1 | chr. 12.502 | AC004466 | DBD + LBD |
| PXR NR1I2 | chr. 3.1354 | AC069444 | DBD + LBD |
| CAR NR1I3 | chr. 1 | AL590714 | DBD + LBD |
| HNF4a NR2A1 | chr. 20.444 | AL132772 | DBD + LBD |
| HNF4g NR2A2 | chr. 8.827 | AC061989.2 | DBD + LBD |
| pseudo-HNF4g | chr. 13 | AL158054 | DBD + LBD |
| RXRα NR2B1 | chr. 9 | AL158031 | DBD + LBD |
| RXRβ NR2B2 | chr. 6.367 | AL031228.1 | DBD + LBD |
| RXRγ NR2B3 | chr. 1.1870 | AC009625.4 | DBD + partial-LBD |
| TR2 NR2C1 | chr. 12 | AC011598 | DBD + LBD |
| TR4 NR2C2 | chr. 3.227 | AC011699.9 | DBD + LBD |
| TLL NR2E1 | chr. 6.1168 | AC027711.2 | DBD + LBD |
| PNR NR2E3 | chr. 7 | AC084299 | DBD + LBD |
| COUPα NR2F1 | chr. 5.1035 | AC008516 | DBD + LBD |
| COUPβ NR2F2 | chr. 15.948 | AC016251 | DBD + LBD |
| EAR2 NE2F6 | chr. 14 | AC010646 | DBD + LBD |
| pseudo-EAR2 | chr. 15.175 | AC020679 | mid-LBD |
| ERα NR3A1 | chr. 6.1623 | AC058817.4 | DBD |
| | chr. 6.1624 | AL049821.6 | mid-LBD |
| | chr. 6.1625 | AL078582.13 | end-LBD |
| ERβ NR3A2 | chr. 14 | CNS01RHJ/ | DBD + LBD |
| | | AL161756 | |
| ERRα NR3B1 | chr. 11.683 | AC005848.1 | DBD + partial-LBD |
| pseudo-ERRα | chr. 13.164 | AC021256.4 | DBD + LBD |
| ERRβ NR3B2 | chr. 14.757 | AC016543.5 | DBD + begining-LBD |
| | chr. 14.758 | AC008050.6 | LBD |
| ERRγ NR3B3 | chr. 1 | AL356008 | DBD + partial LBD |
| | chr. 1 | AL512650 | LBD |
| GR NR3C1 | chr. 5.1617 | AC012634.6 | DBD + LBD |
| MR NR3C2 | chr. 4.1548 | AC010833.3 | DBD + begining-LBD |
| PR NR3C3 | chr. 11 | AP001533 | DBD + LBD |
| AR NR3C4 | chr. X.615 | AL158016.9 | begining-DBD |
| | chr. X.616 | AL158016.9 | end-DBD + LBD |
| NGFIB NR4A1 | chr. 12.549 | AC019244.3 | DBD + LBD |
| NURR1 NR4A2 | chr. 2.1627 | AC073847.1 | DBD + LBD |
| NOR1 NR4A3 | chr. 9.1023 | AC073459.3 | DBD + LBD |
| SF1 NR5A1 | chr. 9.1275 | AL137846 | DBD + LBD |
| LRH1 NR5A2 | chr. 1.2296 | AF190464 | DBD + begining-LBD |
| | chr. 1.2297 | AF190464.1 | mid-LBD |
| | chr. 1.2298 | AC024348.3 | end-LBD |
| GCNF1 NR6A1 | chr. 9.1275 | AL354979 | DBD + LBD |
| SHP NR0B3 | chr. 1.294 | AC004873.2 | LBD |
| DAX NR0B1 | chr. X | AC005185 | LBD |

The above identification of superfamily receptors having DBDs and LDBs by the Human Genome Project provide candidate receptors for the methods of the present invention. It is expected that particularly those genes with no known ligand may be analyzed by the inventive methods to quickly and cheaply screen candidate ligands and ligand analogues.

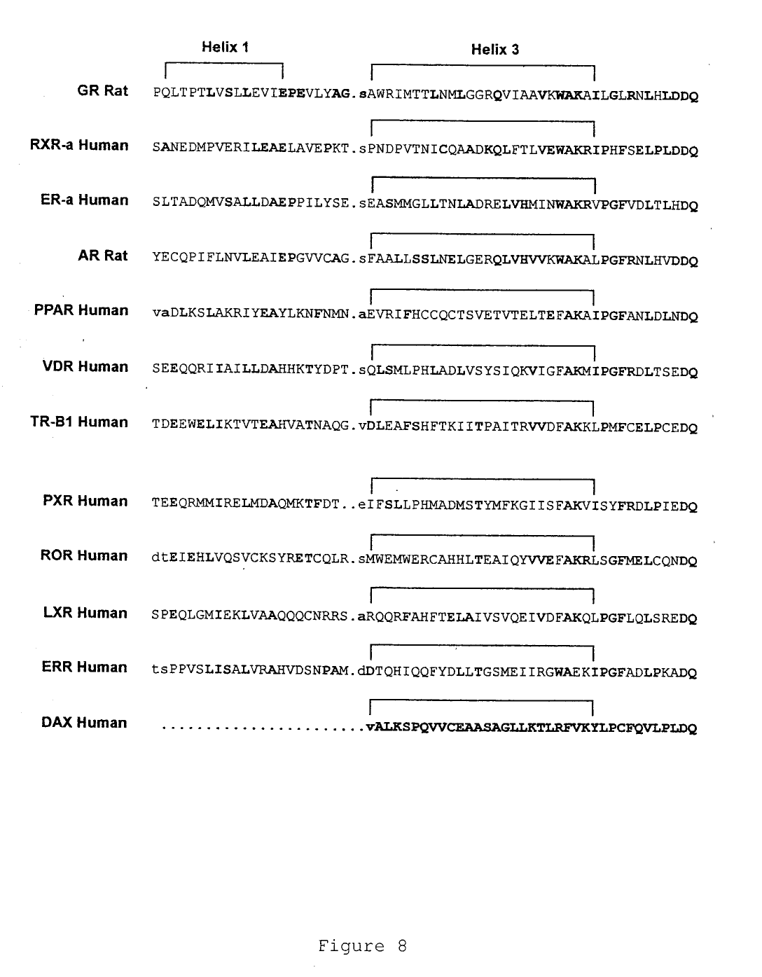

FIG. 8 is a chart which depicts sequence alignments of the protein sequence surrounding helix 1 and helix 3 in several representative mammalian steroid receptors. The sequences in the region of helix 3 demonstrate a relatively high degree of sequence homology, especially when taking into consideration conservative amino acid substitutions. Further, there is even greater functional homology in these domains, given the known and predicted helical tertiary structures in the putative helix 1 and helix 3 domains.

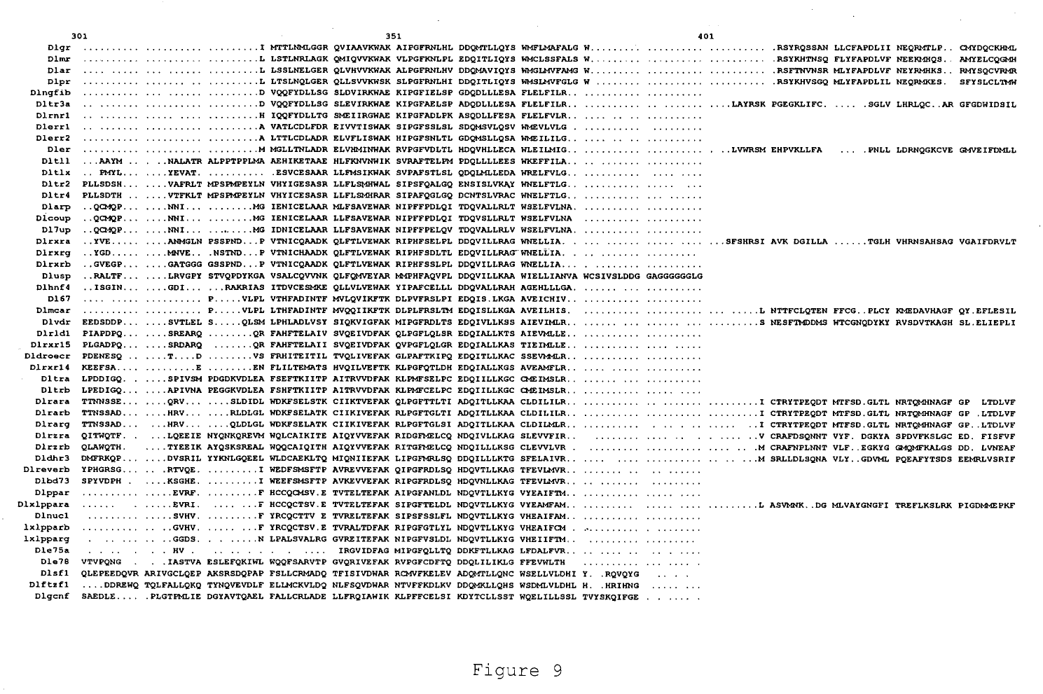

Considering the classic steroid receptor amino sequences shown in FIG. 9, which depicts sequence alignments of the protein sequences surrounding helix 3, it is expected that chimeric molecules may be synthesized from all of these receptors.

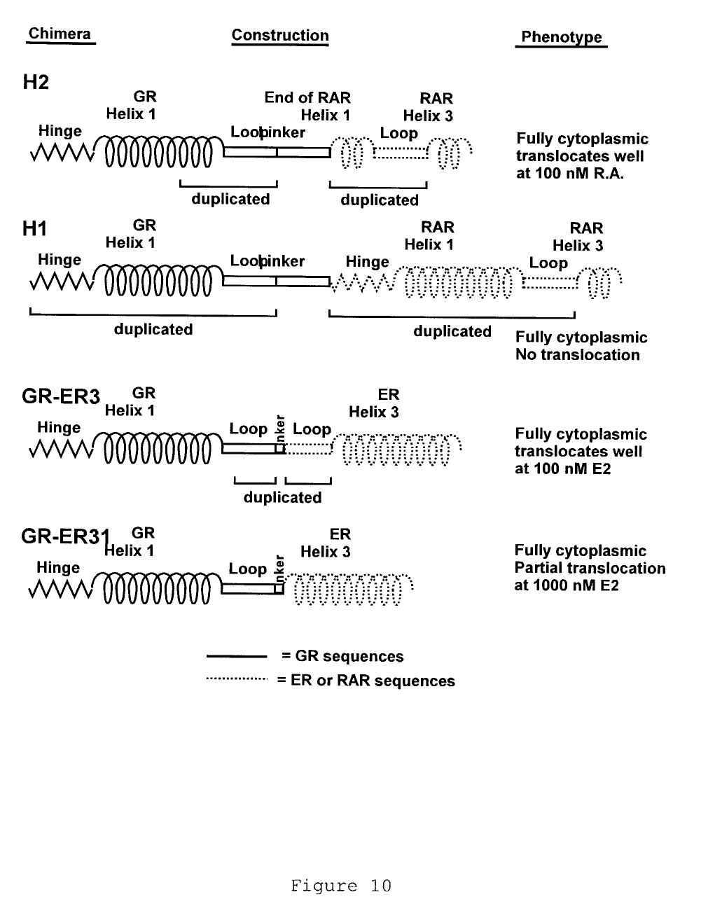

FIG. 10 depicts the synthetic strategy for the synthesis of chimeric GFP-GR-receptor DNA sequences and the corresponding proteins. A GR polypeptide fragment, complete through the DBD, the translocation sequence, and Helix 1, and a linker, such as a multiple cloning site polylinker, are expected to be included in the chimeras. In addition, the LBD, the complete Helix 3 of the desired receptor, and a 5′ linker, for example the ER 1-3 loop, are also expected to be included in the chimeras.

Applicants work indicates that a linker region which is too short, for example by omitting a 5′ linker from the receptor fragment, reduces, but does not eliminate, the translocation efficiency of a chimera. On the other hand, inclusion of a second Helix 1 in the RAR receptor fragment of the chimera, as occurred in GFP-GR-RAR chimera H1 shown in FIG. 10, appears to eliminate the translocation characteristics of that recombinant protein. It is expected that this effect on translocation is a result of changes in the tertiary structure of the recombinant protein produced by a linker region which is simply too long or a result of improper folding of the recombinant protein produced by the presence of the second Helix 1.

As is evident from the present successful results, it is expected that we will successfully generate similar DNA chimeras and corresponding chimeric proteins using other receptor LBDs, and then to identify new ligands and to detect ligand levels in situ. Such chimeras provide a ligand detection tool fundamentally distinct from existing techniques, which test receptor responses at the gene activation, or transcriptional, level. The approach described here tests directly for the ability of an LBD to induce translocation and hence interactions with transcriptional coactivators, chromatin remodeling factors, etc, are unlikely to play a significant role.

### Example 4

Direct Visualization Assays of Ligand Distribution in Living Cells, in Real Time

A GFP-coupled GR-receptor chimera, such as GFP-GR-ER or GFP-GR-RAR, provides the unique capability to directly visualize dynamic, temporo-spatial distributions of superfamily receptors in real time, in live organisms. A GFP-GR-RAR protein is a representative example.

Localization efforts using retinoid-responsive promoters to drive reporter genes are inherently limited by the slow response and level of sensitivity to ligand in co-cultures of reporter cells with tissue explants, or by their dependence on endogenous cellular receptors, cofactors, and the limitations of the particular transgenic promoters used. In contrast, the inventive translocation based assay requires only about 20-30 minutes of exposure, is visualized directly in vivo, and is independent of the particular transcriptional milieu of the cell.

Screens for novel receptor ligands using transcriptional reporters were previously similarly limited, whereas the translocation assay with a GFP-tagged chimera requires no biochemical manipulations and allows rapid, direct visualization in living cells. Epifluorescent cellular imaging systems driven by artificial intelligence software can now automatically scan and quantify the subcellular distribution of fluorescent molecules in large numbers of living cell pools. These imaging systems, coupled with the inventive translocation assay, can be employed in high-throughput screens for novel receptor ligands in vivo.

It is expected that the inventive translocation assays will provide equal or better sensitivity, at lower cost, with higher throughput rates, and with quicker processing times when compared to other assays known in the art.

The invention being thus described, it will be obvious that the same may be modified or varied in many ways. Such modifications and variations are not to be regarded as a departure from the spirit and scope of the invention and all such modifications and variations are intended to be included within the scope of the following claims.

We claim:

1. A method for making a recombinant nuclear translocation protein, comprising:
covalently connecting (i) a glucocorticoid receptor DNA sequence coding for the cytoplasmic/nuclear translocation domain of the glucocorticoid receptor protein, (ii) a superfamily receptor DNA sequence coding for the ligand binding domain of a superfamily receptor protein, and (iii) a nucleic acid sequence for a marker protein domain, to form a DNA chimera,
wherein said superfamily receptor DNA sequence is connected to the 3′ end of said glucocorticoid receptor DNA sequence; and
expressing said DNA chimera in an expression system to prepare said protein.

2. The method of claim 1, further comprising:
covalently connecting said translocation domain of the glucocorticoid receptor and said ligand binding domain of a superfamily receptor utilizing a DNA linker sequence.

3. The method of claim 2, wherein said DNA linker sequence is a fragment of a polylinker sequence.

4. The method of claim 1, wherein said marker protein domain DNA sequence is covalently connected to the 5′ end of said glucocorticoid receptor DNA sequence.

5. The method of claim 4, further comprising:
covalently connecting said glucocorticoid receptor DNA sequence and said nucleic acid sequence for a marker protein utilizing a DNA linker sequence.

6. The method of claim 1, wherein said glucocorticoid receptor DNA sequence encompasses the 5′ end of said sequence through and including the complete nuclear localization sequence of said glucocorticoid receptor DNA sequence.

7. The method of claim 1, wherein said glucocorticoid receptor DNA sequence encompasses the 5′ end of said sequence through and including the complete nuclear localization sequence and the complete helix 1 sequence of said glucocorticoid receptor DNA sequence.

8. The method of claim 1, wherein said glucocorticoid receptor DNA sequence encompasses the 5′ end of said sequence through and including DNA bases corresponding to about amino acid residue 570of said glucocorticoid receptor protein.

9. The method of claim 1, wherein said superfamily receptor DNA sequence encompasses the 3′ end of said sequence through and including the complete ligand binding domain sequence and the complete helix 3 sequence of said superfamily receptor DNA sequence.

10. The method of claim 1, wherein said superfamily receptor DNA sequence encompasses the 3′ end of said sequence through and including the complete ligand binding domain sequence, the complete helix 3 sequence, and at most a fragment of helix 1 of said superfamily receptor DNA sequence.

11. A protein produced by the process of:
covalently connecting (i) a glucocorticoid receptor DNA sequence coding for the 5′ end of the DNA sequence of the glucocorticoid receptor protein, through and including the complete nuclear localization sequence and the complete helix 1 sequence of said glucocorticoid receptor DNA sequence, (ii) a superfamily receptor DNA sequence coding for the 3′ end of the DNA sequence of a superfamily receptor protein, through and including the complete ligand binding domain sequence and the complete helix 3 sequence of said superfamily receptor DNA sequence, and (iii) a nucleic acid sequence for a marker protein domain, to form a DNA chimera,
wherein said superfamily receptor DNA sequence is connected to the 3′ end of said glucocorticoid receptor DNA sequence,
wherein said marker protein domain DNA sequence is covalently connected to the 5′ end of said glucocorticoid receptor DNA sequence,
and wherein said translocation domain of the glucocorticoid receptor and said ligand binding domain of a superfamily receptor are covalently connected by a DNA linker sequence; and
expressing said DNA chimera in an expression system to prepare said protein.

12. A nucleic acid chimera comprising:
a nucleic acid sequence which codes for the cytoplasmic/nuclear translocation domain of glucocorticoid receptor protein; and
a nucleic acid sequence which codes for the ligand binding domain of a superfamily receptor protein.

13. The nucleic acid chimera of claim 12, additionally comprising:
a nucleic acid sequence for a marker protein domain.

14. The nucleic acid chimera of claim 13, wherein said marker protein domain encodes a fluorescent protein.

15. The nucleic acid chimera of claim 12, wherein said glucocorticoid receptor DNA sequence encompasses the 5′ end of said sequence through and including the complete nuclear localization sequence of said glucocorticoid receptor DNA sequence.

16. The nucleic acid chimera of claim 12, wherein said glucocorticoid receptor DNA sequence encompasses the 5′ end of said sequence through and including the complete nuclear localization sequence and the complete helix 1 sequence of said glucocorticoid receptor DNA sequence.

17. The nucleic acid chimera of claim 12, wherein said glucocorticoid receptor DNA sequence encompasses the 5′ end of said sequence through and including DNA bases corresponding to about amino acid residue 570of said glucocorticoid receptor protein.

18. The nucleic acid chimera of claim 12, wherein said superfamily receptor DNA sequence encompasses the 3′ end of said sequence through and including the complete ligand binding domain sequence and the complete helix 3 sequence of said superfamily receptor DNA sequence.

19. The nucleic acid chimera of claim 12, wherein said superfamily receptor DNA sequence encompasses the 3′ end of said sequence through and including the complete ligand binding domain sequence, the complete helix 3 sequence, and at most a fragment of helix 1 of said superfamily receptor DNA sequence.

20. The nucleic acid chimera of claim 12, wherein said ligand binding domain is the ligand binding domain of estrogen receptor.

21. The nucleic acid chimera of claim 12, which is SEQ. ID NO. 1.

22. The nucleic acid chimera of claim 12, wherein said ligand binding domain is the ligand binding domain of retinoic acid receptor.

23. The nucleic acid chimera of claim 12, which is SEQ. ID NO. 2.

24. A chimeric protein comprising two elements:
a glucocorticoid receptor 5′ end, encompassing the nuclear translocation domain and helix 1; and
a superfamily receptor 3′ end, encompassing the ligand binding domain and helix 3.

25. The chimeric protein of claim 24, further comprising a marker protein domain.

26. The chimeric protein of claim 25, wherein said marker protein domain encodes a fluorescent protein.

27. The chimeric protein of claim 24, wherein said ligand binding domain is the ligand binding domain of estrogen receptor.

28. The chimeric protein of claim 24, which is SEQ. ID NO. 3.

29. The chimeric protein of claim 24, wherein said ligand binding domain is the ligand binding domain of retinoic acid receptor.

30. The chimeric protein of claim 24, which is SEQ. ID NO. 4.

31. A method for detecting a ligand of a superfamily receptor protein, which comprises:
producing a nucleic acid vector encoding a nucleic acid chimera comprising three elements: a 5′ end of a glucocorticoid receptor, encompassing the nuclear translocation domain and helix 1, a 3′ end of a superfamily receptor, encompassing the ligand binding domain and helix 3, and a nucleic acid sequence for a marker protein domain;
transfecting a eukaryotic cell with said nucleic acid vector;
isolating a clonal population of cells that express a chimeric protein translated from said nucleic acid vector;
contacting said cells with a sample compound or composition; and
detecting the presence of cytoplasmic/nuclear translocation in response to a ligand of said ligand binding domain.

32. The method of claim 31, wherein said marker protein domain encodes a fluorescent protein.

33. A method for determining the concentration of a ligand of a labeled chimeric superfamily receptor protein, which comprises:
producing a nucleic acid vector encoding a nucleic acid chimera comprising: a nucleic acid sequence coding for the cytoplasmic/nuclear translocation domain of glucocorticoid receptor protein, a nucleic acid sequence coding for the ligand binding domain of a superfamily receptor protein, and a nucleic acid sequence for a marker protein domain;
transfecting a eukaryotic cell with said nucleic acid vector;
isolating a clonal population of transfected cells that express a chimeric protein translated from said nucleic acid vector;
contacting said transfected cells with a sample;
scanning one or more test cell(s) to obtain signal data from said labeled protein;
converting said signal data to obtain the cellular location of said labeled protein in said test cell(s); and
analyzing said data using an analysis system having an algorithm to calculate changes in the distribution of said labeled protein between the cell cytoplasm and the cell nucleus of said test cell(s), said analysis system having the capability of providing an accurate reading of the concentration of a ligand.

34. The method of claim 33, wherein said marker protein domain encodes a fluorescent protein.

35. A kit for detecting and screening for a ligand of a superfamily receptor protein in an environmental sample, comprising:
a cell-based system which expresses a chimeric protein comprising the cytoplasmic/nuclear translocation domain of glucocorticoid receptor protein, the ligand binding domain of a superfamily receptor protein, and a marker protein domain; and
a detection system for the detection of the translocation of said marker protein.

36. The kit of claim 35, additionally comprising:
one or more compounds and/or compositions which stably associate with said chimeric protein in the absence of a ligand for the ligand binding domain of said chimeric protein, and which dissociates from said chimeric protein in the presence of a ligand for the ligand binding domain of said chimeric protein.

37. A kit for detecting and screening for a ligand of a superfamily receptor protein in an environmental sample, comprising:
a quantity of a chimeric protein comprising the cytoplasmic/nuclear translocation domain of glucocorticoid receptor protein, the ligand binding domain of a superfamily receptor protein, and a marker protein domain;
a cell-free membrane system which restricts translocation of the chimeric protein when no ligand is bound to the ligand binding domain of said chimeric protein, and which permits translocation of the chimeric protein when the ligand binding domain of said chimeric protein is bound to its ligand; and
a detection system for the detection of the translocation of said marker protein.

38. The kit of claim 37, additionally comprising:
one or more compounds and/or compositions which stably associate with said chimeric protein in the absence of a ligand for the ligand binding domain of said chimeric protein, and which dissociates from said chimeric protein in the presence of a ligand for the ligand binding domain of said chimeric protein.

39. A method for diagnosis of defects in the nuclear transportation process, which comprises:
producing a nucleic acid vector encoding a nucleic acid chimera comprising: a nucleic acid sequence coding for the cytoplasmic/nuclear translocation domain of glucocorticoid receptor protein, a nucleic acid sequence coding for the ligand binding domain of a superfamily receptor protein, and a nucleic acid sequence for a marker protein domain;
transfecting a set of suspected defective cells with said nucleic acid vector;
isolating a clonal population of said cells that express a chimeric protein translated from said nucleic acid vector;
contacting said cells with a ligand of said ligand binding domain; and
detecting the presence or absence of cytoplasmic/nuclear translocation in response to said ligand.

40. A method for treating defective translocation of a superfamily receptor protein from the cytoplasm to the nucleus of a cell, in an animal in need thereof, comprising:
producing a nucleic acid vector which is capable of being transcribed, and which encodes a nucleic acid chimera comprising: a nucleic acid sequence coding for the cytoplasmic/nuclear translocation domain of glucocorticoid receptor protein and a nucleic acid sequence coding for the ligand binding domain of said superfamily receptor protein; and
transfecting a target cell in said animal with said nucleic acid vector.

41. A pharmaceutical composition comprising:
(i) a chimeric protein comprising two elements:
a glucocorticoid receptor 5′ end, encompassing the nuclear translocation domain and helix 1; and
a superfamily receptor 3′ end, encompassing the ligand binding domain and helix 3; and
(ii) a pharmaceutically acceptable carrier.


SEQUENCE LISTING (6 sequences)

SEQ ID NO: 1 - protein, 1147 aa, Rat/human chimera
  mat_peptide (1)..(): Chimeric Protein
MSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGAGAGAGAGAISALLDSKESLAPPGRDEVPGSLLGQGRGSVMDFYKSLRGGATVKVSASSPSVAAASQADSKQQRILLDFSKGSTSNVQQRQQQQQQQQQQQQQQQQQQQPGLSKAVSLSMGLYMGETETKVMGNDLGYPQQGQLGLSSGETDFRLLEESIANLNRSTSVPENPKSSTSATGCATPTEKEFPKTHSDASSEQQNRKSQTGTNGGSVKLYPTDQSTFDLLKDLEFSAGSPSKDTNESPWRSDLLIDENLLSPLAGEDDPFLLEGNTNEDCKPLILPDTKPKIKDTGDTILSSPSSVALPQVKTEKDDFIELCTPGVIKQEKLGPVYCQASFSGTNIIGNKMSAISVHGVSTSGGQMYHYDMNTASLSQQQDQKPVFNVIPPIPVGSENWNRCQGSGEDSLTSLGALNFPGRSVFSNGYSSPGMRPDVSSPPSSSSAATGPPPKLCLVCSDEASGCHYGVLTCGSCKVFFKRAVEGQHNYLCAGRNDCIIDKIRRKNCPACRYRKCLQAGMNLEARKTKKKIKGIQQATAGVSQDTSENPNKTIVPAALPQLTPTLVSLLEVIEPEVLYAGYDSSSRGEPPILYSEYDPTRPFSEASMMGLLTNEPEVLYAGYDSSVSRGLSSKAHQETFPALCQLGKYTTNNSSEQRVSLDIDLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 2 - protein, 1089 aa, Rat/human chimera
  mat_peptide (1)..(): Chimeric Protein
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGALLDSKESLAPPGRDEVPGSLLGQGRGSVMDFYKSLRGGATVKVSASSPSVAAASQADSKQQRILLDFSKGSTSNVQQRQQQQQQQQQQQQQQQQQQQPGLSKAVSLSMGLYMGETETKVMGNDLGYPQQGQLGLSSGETDFRLLEESIANLNRSTSVPENPKSSTSATGCATPTEKEFPKTHSDASSEQQNRKSQTGTNGGSVKLYPTDQSTFDLLKDLEFSAGSPSKDTNESPWRSDLLIDENLLSPLAGEDDPFLLEGNTNEDCKPLILPDTKPKIKDTGDTILSSPSSVALPQVKTEKDDFIELCTPGVIKQEKLGPVYCQASFSGTNIIGNKMSAISVHGVSTSGGQMYHYDMNTASLSQQQDQKPVFNVIPPIPVGSENWNRCQGSGEDSLTSLGALNFPGRSVFSNGYSSPGMRPDVSSPPSSSSAATGPPPKLCLVCSDEASGCHYGVLTCGSCKVFFKRAVEGQHNYLCAGRNDCIIDKIRRKNCPACRYRKCLQAGMNLEARKTKKKIKGIQQATAGVSQDTSENPNKTIVPAALPQLTPTLVSLLEVIEPEVLYAGYDSSVSRGLSSKAHQETFPALCQLGKYTTNNSSEQRVSLDIDLWDKFSELSTKCIIKTVEFAKQLPGFTTLTIADQITLLKAACLDILILRICTRYTPEQDTMTFSDGLTLNRTQMHNAGFGPLTDLVFAFANQLLPLEMDDAETGLLSAICLICGDRQDLEQPDRVDMLQEPLLEALKVYVRKRRPSRPHMFPKMLMKITDLRSISAKGAERVITLKMEIPGSMPPLIQEMLENSEGLDTLSGQPGGGGRDGGGLAPPPGSCSPSLSPSSNRSSPATHSP

SEQ ID NO: 3 - DNA, 3300 bp, Rat/human Chimera
  CDS (1)..(3300)
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttcacttatggtgttcaatgcttttcaagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaggcgccggcgctggtgctggtgctggcgccatcagcgcgctgatcctggactccaaagaatccttagctccccctggtagagacgaagtccctggcagtttgcttggccaggggagggggagcgtaatggacttttataaaagcctgaggggaggagctacagtcaaggtttctgcatcttcgccctcagtggctgctgcttctcaggcagattccaagcagcagaggattctccttgatttctcgaaaggctccacaagcaatgtgcagcagcgacagcagcagcagcagcagcagcagcagcagcagcagcagcagcagcagcagcagcagccaggcttatccaaagccgtttcactgtccatggggctgtatatgggagagacagaaacaaaagtgatggggaatgacttgggctacccacagcagggccaacttggcctttcctctggggaaacagactttcggcttctggaagaaagcattgcaaacctcaataggtcgaccagcgttccagagaaccccaagagttcaacgtctgcaactgggtgtgctaccccgacagagaaggagtttcccaaaactcactcggatgcatcttcagaacagcaaaatcgaaaaagccagaccggcaccaacggaggcagtgtgaaattgtatcccacagaccaaagcacctttgacctcttgaaggatttggagttttccgctgggtccccaagtaaagacacaaacgagagtccctggagatcagatctgttgatagatgaaaacttgctttctcctttggcgggagaagatgatccattccttctcgaagggaacacgaatgaggattgtaagcctcttattttaccggacactaaacctaaaattaaggatactggagatacaatcttatcaagtcccagcagtgtggcactaccccaagtgaaaacagaaaaagatgatttcattgaactttgcacccccggggtaattaagcaagagaaactgggcccagtttattgtcaggcaagcttttctgggacaaatataattggtaataaaatgtctgccatttctgttcatggtgtgagtacctctggaggacagatgtaccactatgacatgaatacagcatccctttctcagcagcaggatcagaagcctgtttttaatgtcattccaccaattcctgttggttctgaaaactggaataggtgccaaggctccggagaggacagcctgacttccttgggggctctgaacttcccaggccggtcagtgttttctaatgggtactcaagccctggaatgagaccagatgtaagctctcctccatccagctcgtcagcagccacgggaccacctcccaagctctgcctggtgtgctccgatgaagcttcaggatgtcattacggggtgctgacatgtggaagctgcaaagtattctttaaaagagcagtggaaggacagcacaattacctttgtgctggaagaaacgattgcatcattgataaaattcgaaggaaaaactgcccagcatgccgctatcggaaatgtcttcaggctggaatgaaccttgaagctcgaaaaacaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgtttcgcgaggtgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctga

SEQ ID NO: 4 - protein, 1099 aa, Rat/human Chimera
MSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGAGAGAGAGAISALILDSKESLAPPGRDEVPGSLLGQGRGSVMDFYKSLRGGATVKVSASSPSVAAASQADSKQQRILLDFSKGSTSNVQQRQQQQQQQQQQQQQQQQQQQPGLSKAVSLSMGLYMGETETKVMGNDLGYPQQGQLGLSSGETDFRLLEESIANLNRSTSVPENPKSSTSATGCATPTEKEFPKTHSDASSEQQNRKSQTGTNGGSVKLYPTDQSTFDLLKDLEFSAGSPSKDTNESPWRSDLLIDENLLSPLAGEDDPFLLEGNTNEDCKPLILPDTKPKIKDTGDTILSSPSSVALPQVKTEKDDFIELCTPGVIKQEKLGPVYCQASFSGTNIIGNKMSAISVHGVSTSGGQMYHYDMNTASLSQQQDQKPVFNVIPPIPVGSENWNRCQGSGEDSLTSLGALNFPGRSVFSNGYSSPGMRPDVSSPPSSSSAATGPPPKLCLVCSDEASGCHYGVLTCGSCKVFFKRAVEGQHNYLCAGRNDCIIDKIRRKNCPACRYRKCLQAGMNLEARKTKKKIKGIQQATAGVSQDTSENPNKTIVPAALPQLTPTLVSLLEVIEPEVLYAGYDSSVSRGEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 5 - DNA, 3273 bp, Rat/human Chimera
  CDS (1)..(3273)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggcgcgctgatcctggactccaaagaatccttagctccccctggtagagacgaagtccctggcagtttgcttggccaggggagggggagcgtaatggacttttataaaagcctgaggggaggagctacagtcaaggtttctgcatcttcgccctcagtggctgctgcttctcaggcagattccaagcagcagaggattctccttgatttctcgaaaggctccacaagcaatgtgcagcagcgacagcagcagcagcagcagcagcagcagcagcagcagcagcagcagcagcagcagcagccaggcttatccaaagccgtttcactgtccatggggctgtatatgggagagacagaaacaaaagtgatggggaatgacttgggctacccacagcagggccaacttggcctttcctctggggaaacagactttcggcttctggaagaaagcattgcaaacctcaataggtcgaccagcgttccagagaaccccaagagttcaacgtctgcaactgggtgtgctaccccgacagagaaggagtttcccaaaactcactcggatgcatcttcagaacagcaaaatcgaaaaagccagaccggcaccaacggaggcagtgtgaaattgtatcccacagaccaaagcacctttgacctcttgaaggatttggagttttccgctgggtccccaagtaaagacacaaacgagagtccctggagatcagatctgttgatagatgaaaacttgctttctcctttggcgggagaagatgatccattccttctcgaagggaacacgaatgaggattgtaagcctcttattttaccggacactaaacctaaaattaaggatactggagatacaatcttatcaagtcccagcagtgtggcactaccccaagtgaaaacagaaaaagatgatttcattgaactttgcacccccggggtaattaagcaagagaaactgggcccagtttattgtcaggcaagcttttctgggacaaatataattggtaataaaatgtctgccatttctgttcatggtgtgagtacctctggaggacagatgtaccactatgacatgaatacagcatccctttctcagcagcaggatcagaagcctgtttttaatgtcattccaccaattcctgttggttctgaaaactggaataggtgccaaggctccggagaggacagcctgacttccttgggggctctgaacttcccaggccggtcagtgttttctaatgggtactcaagccctggaatgagaccagatgtaagctctcctccatccagctcgtcagcagccacgggaccacctcccaagctctgcctggtgtgctccgatgaagcttcaggatgtcattacggggtgctgacatgtggaagctgcaaagtattctttaaaagagcagtggaaggacagcacaattacctttgtgctggaagaaacgattgcatcattgataaaattcgaaggaaaaactgcccagcatgccgctatcggaaatgtcttcaggctggaatgaaccttgaagctcgaaaaacaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgtttcgcgaggcctctcgagcaaagcgcaccaggaaaccttccctgccctctgccagctgggcaaatacactacgaacaacagctcagaacaacgtgtctctctggacattgacctctgggacaagttcagtgaactctccaccaagtgcatcattaagactgtggagttcgccaagcagctgcccggcttcaccaccctcaccatcgccgaccagatcaccctcctcaaggctgcctgcctggacatcctgatcctgcggatctgcacgcggtacacgcccgagcaggacaccatgaccttctcggacgggctgaccctgaaccggacccagatgcacaacgctggcttcggccccctcaccgacctggtctttgccttcgccaaccagctgctgcccctggagatggatgatgcggagacggggctgctcagcgccatctgcctcatctgcggagaccgccaggacctggagcagccggaccgggtggacatgctgcaggagccgctgctggaggcgctaaaggtctacgtgcggaagcggaggcccagccgcccccacatgttccccaagatgctaatgaagattactgacctgcgaagcatcagcgccaagggggctgagcgggtgatcacgctgaagatggagatcccgggctccatgccgcctctcatccaggaaatgttggagaactcagagggcctggacactctgagcggacagccggggggtggggggcgggacgggggtggcctggcccccccgccaggcagctgtagccccagcctcagccccagctccaacagaagcagcccggccacccactccccgtga

SEQ ID NO: 6 - protein, 1090 aa, Rat/human Chimera
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGALILDSKESLAPPGRDEVPGSLLGQGRGSVMDFYKSLRGGATVKVSASSPSVAAASQADSKQQRILLDFSKGSTSNVQQRQQQQQQQQQQQQQQQQQQQPGLSKAVSLSMGLYMGETETKVMGNDLGYPQQGQLGLSSGETDFRLLEESIANLNRSTSVPENPKSSTSATGCATPTEKEFPKTHSDASSEQQNRKSQTGTNGGSVKLYPTDQSTFDLLKDLEFSAGSPSKDTNESPWRSDLLIDENLLSPLAGEDDPFLLEGNTNEDCKPLILPDTKPKIKDTGDTILSSPSSVALPQVKTEKDDFIELCTPGVIKQEKLGPVYCQASFSGTNIIGNKMSAISVHGVSTSGGQMYHYDMNTASLSQQQDQKPVFNVIPPIPVGSENWNRCQGSGEDSLTSLGALNFPGRSVFSNGYSSPGMRPDVSSPPSSSSAATGPPPKLCLVCSDEASGCHYGVLTCGSCKVFFKRAVEGQHNYLCAGRNDCIIDKIRRKNCPACRYRKCLQAGMNLEARKTKKKIKGIQQATAGVSQDTSENPNKTIVPAALPQLTPTLVSLLEVIEPEVLYAGYDSSVSRGLSSKAHQETFPALCQLGKYTTNNSSEQRVSLDIDLWDKFSELSTKCIIKTVEFAKQLPGFTTLTIADQITLLKAACLDILILRICTRYTPEQDTMTFSDGLTLNRTQMHNAGFGPLTDLVFAFANQLLPLEMDDAETGLLSAICLICGDRQDLEQPDRVDMLQEPLLEALKVYVRKRRPSRPHMFPKMLMKITDLRSISAKGAERVITLKMEIPGSMPPLIQEMLENSEGLDTLSGQPGGGGRDGGGLAPPPGSCSPSLSPSSNRSSPATHSP